(19) United States
(12) Patent Application Publication
(10) Pub. No.: US 2026/0248735 A1
(43) Pub. Date: Aug. 27, 2026

(54) A METHOD FOR PREPARING MICROPARTICLES FOR DRUG DELIVERY

(71) Applicant: Danmarks Tekniske Universitet, Kongens Lyngby (DK)

(72) Inventors: Lasse Højlund Eklund Thamdrup, Kongens Lyngby (DK); Mette Schnoor Mosgaard, Kongens Lyngby (DK); Line Hagner Nielsen, Kongens Lyngby (DK); Mahdi Ghavami, Kongens Lyngby (DK); Carmen Milian Guimera, Kongens Lyngby (DK); Anja Boisen, Kongens Lyngby (DK)

(21) Appl. No.: 19/161,062

(22) PCT Filed: Feb. 29, 2024
(86) PCT No.: PCT/EP2024/055262

§ 371 Date: Aug. 29, 2025

(30) Foreign Application Priority Data
Mar. 3, 2023 (EP) 23159956.4

(51) Int. Cl. A61K 9/50; A61K 31/405; A61K 38/23; A61K 49/04

(52) U.S. Cl. CPC A61K 9/5089; A61K 9/5026; A61K 9/5031; A61K 31/405; A61K 38/23; A61K 49/0404; A61K 49/0419

(57) ABSTRACT

Disclosed is a method for preparing microparticles for drug delivery. The method comprises providing a mold comprising multiple individual, open compartments arranged on a first surface of the mold. The open compartments are separated from each other by one or more edges. Each edge has a width of 5 μm or less. The method comprises coating the first surface of the mold with a mold layer comprising a biocompatible and/or biodegradable material having a first release characteristic at a first condition. The method comprises loading the compartments coated with the mold layer with a pharmaceutical composition comprising a drug substance. The method comprises coating the first surface of the mold with a top layer comprising a biocompatible and/or biodegradable material having a second release characteristic at the first condition for provision of closed microparticles in the multiple individual compartments. The method comprises separating the closed microparticles by cutting the mold layer and the top layer using the one or more edges. By cutting the mold layer and the top layer using the one or more edges discrete microparticles are provided.

The present disclosure pertains to the field of pharmaceutical compositions/technology. The present disclosure relates to a method for producing microparticles, such as monodisperse non-spherical microparticles, that are loaded with a pharmaceutical composition. In particular, it relates to a method that is capable of mass producing identical microparticles in which a relevant pharmaceutical composition is encapsulated inside a polymer shell that can be composed of one or more materials.

### BACKGROUND

Over the course of the last two decades there has been an increasing interest in producing and utilizing microscale particles and devices for delivery of pharmaceutical compositions. For some administration routes, such as the nasal and pulmonary route, microscopic particles, having sizes of approximately 100 μm or below, are explicitly required to ensure delivery at the intended target site. For other administration routes including oral, buccal, sublingual, ocular, rectal and vaginal routes, it is no explicit requirement that the particle size is in the sub-100 μm regime. There are empirical indications from pre-clinical and clinical experiments that utilizing microencapsulated pharmaceutical compositions can increase the drug uptake and/or absorption. In the specific case of oral delivery, the increased absorption is believed to be the result of synergetic effects associated with i) increased retention of the drug particles, ii) stabilization of the active pharmaceutical ingredients (APIs), iii) increased drug dissolution, and iv) enhanced unidirectional release in close proximity of the epithelium. Utilizing microencapsulated pharmaceutical compositions is currently mostly aimed at Biopharmaceutical Classification System (BCS) class 2-4 drugs where the limited uptake/absorption are associated with poor solubility and/or poor permeability. There is limited empirical data that suggests that microencapsulation in itself can enhance permeability. However, it is speculated that the increased particle retention and drug solubility in conjunction with close proximity to the epithelium can enhance uptake/absorption. For the delivery of pharmaceutical compositions that are prone to acidic or enzymatic degradation, microencapsulation can be used to protect the API prior to release and absorption. This is especially beneficial when considering sensitive substances such as vaccines, peptides and proteins that are often quite expensive to produce.

Conventional microencapsulation methods include freeze drying, spray drying, spray cooling, fluidized bed coating, coacervation, emulsification and coextrusion. Said methods are employed extensively for producing encapsulated pharmaceutical compositions. The produced particles will exhibit spherical or perturbed spherical shapes where the mean diameter can be tuned in the range from approximately 1 μm to several hundred micrometers. The conventional methods allow readily for producing polydisperse microparticles where the loaded API is released omnidirectionally in response to dissolution or erosion of the encapsulating material. From a purely aerodynamic and/or hydrodynamic point of view, the spherical morphology is attractive as it enhances flyability and reduces hydrodynamic drag which can be attractive for pulmonary delivery and parenteral delivery where increased bloodstream circulation is needed. However, the shape is less favorable for e.g., oral administration as here, ensuring adhesion to or engulfment inside mucus can increase oral bioavailability. In addition, the omnidirectional release and the limited surface-area-to-volume ratio of spherical particles are considered less feasible when it comes to especially oral delivery of APIs. Finally, the polydisperse nature of particles produced using conventional methods can be problematic in terms of ensuring optimal delivery via especially the nasal and pulmonary routes.

### SUMMARY

Accordingly, there is a need for a drug delivery solution, which mitigates, alleviates or addresses the existing shortcomings and provides an improved release and/or absorption of the drug.

Disclosed is a method for preparing microparticles for drug delivery. The method relies on a microfabricated mold comprising multiple individual, open compartments arranged on a first surface of the mold. The open compartments are separated from each other by one or more edges. Each edge has a width of 5 μm or less. The method comprises coating the first surface of the mold with a mold layer comprising a biocompatible and/or biodegradable material having a first release characteristic at a first condition. The method comprises loading the compartments coated with the mold layer with a pharmaceutical composition comprising a drug substance. The method comprises coating the first surface of the mold with a top layer comprising a material having a second release characteristic at the first condition for provision of closed microparticles. The method comprises separating the individual, closed microparticles by cutting the mold layer and the top layer using the one or more edges, for provision of monodisperse discrete microparticles.

It is an advantage of the mold of the current disclosure that the edge separating individual compartments has a narrow width, which together with the shape of the compartments creates a cutting edge, which makes it easy to cut the mold layer and the top layer of the filled compartments to obtain monodisperse microparticles while avoiding superfluous material to discard. By applying a force or a pressure to the backside of the mold or conversely to the top layer of the sealed microparticles, the edges, such as the cutting edges, separating the compartments of the mold penetrate the mold layer and the top layer, so that the loaded microparticles are separated from each other. Moreover, by providing the mold layer and the top layer of the particles with different release characteristics, a predetermined and controlled release of the pharmaceutical composition from the microparticles can be obtained. The method provided herein allows monodisperse particles having non-spherical morphologies to be produced, that allow for triggered unidirectional release. The current disclosure introduces a new fabrication method which allows for mass-producing monodisperse microencapsulated solid or semisolid state APIs using conventional coating methods in conjunction with room temperature particle formation. The method presented in the current application thus alleviates drug loading, is compatible with microencapsulation of solvent and/or temperature sensitive APIs. Furthermore, the method allows for tuning the shape, size, release profile and adhesive properties of the produced particles.

### DEFINITIONS AND DESCRIPTION OF TERMS USED HEREIN

In the present disclosure, the term “close-packed” refers to an arrangement of the compartments on a microfabricated mold, such as on a mold. More specifically, “close-packed” entails that neighboring compartments are separated only by the protruding sidewalls which effectively means that the entire surface of the mold is used for producing microparticles. This in turn entails that only geometries that allow for regular tessellation with no overlaps or voids will be perceived as suitable to achieve a close-packed array of compartments.

In the present disclosure, the term “positively tapered” refers to sidewalls where the angle (shown in FIG. 1) is between 0° and 90°. Hence for ⊖=0° the mold surface is essentially flat and for ⊖=90° the sidewalls are perpendicular to the bottom surface. For ⊖>90°, the sidewalls are “negatively tapered” which prohibits the production of microparticles.

In the present disclosure, the term “conformal” pertains to potential thickness variations of the mold layer that is deposited on the microfabricated mold. A conformal coating is characterized by limited variations when comparing the thickness on horizontal and tapered surfaces.

In the present disclosure, the term “composition” or “pharmaceutical composition” refers to a formulation consisting of one or more active substances intended for conferring a deterministic physiological effect in a living organism. As used herein, the term refers to drugs and vaccines, but it can also refer to probiotics which in this context comprises a microorganism that exerts a beneficial effect on the health of the host to which the probiotic is administered. Additionally, the pharmaceutical composition can comprise substances such as, but not limited to, inhibitors and/or permeation enhancers that are aimed at improving the physiological effect of the active substance or substances in said composition.

In the present disclosure, the term “mold layer” refers to the layer that is directly in contact with the microfabricated mold used for producing the microparticles containing the pharmaceutical composition. The mold layer may be composed of one or more sublayers each comprising one or more materials.

In the present disclosure, the term “top layer” refers to the layer that is coated or deposited on top of the pharmaceutical composition after having loaded the composition into the microcompartments which have been coated with the mold layer. The top layer may be composed of one or more sublayers each comprising one or more materials.

In the present disclosure, the terms “cutting” or “micro-cutting” refers to the processing step in which the microparticles are mechanically cut into discrete entities. Cutting, as opposed to e.g., hot embossing, is a process characterized by a short cycle time and it is conducted at room temperature.

In the present disclosure, the term “ultrasonic spray coating” refers to a deposition or coating method where a low viscosity solution of a material is feed to a dedicated nozzle which allows for atomization of said solution thereby generating a fine mist of solvent droplets with a narrow size distribution where the mean diameter is largely determined by the ultrasonic frequency of the nozzle. The kinetic energy and directionality of the droplets can be fine-tuned by controlling the properties of the air cladding layer.

In the present disclosure, the term “monodisperse” relates to the spatial size of an ensemble of entities. More specifically, an ensemble of particles, grains etc. is said to be monodisperse if the particle size and/or the volume of the particles are similar. This can be measure of that the particle size distribution is narrow, which in turn means that the constituent entities have approximately the same size, or it can be measured by SEM or by volume. For example, the size of the monodisperse particles may span from −10% of the mean particle size to ±10% of the mean particle size such as, e.g., ±5% or ±2.5% of the mean particle size. If the monodispersity is measured by SEM the particles defined by a specific parameter such as the length of the largest dimension, then the length may span from −10% of the mean particle size to ±10% of the mean particle size such as, e.g., ±5% or ±2.5% of the mean particle size. If the monodispersity is measured by volume, the volume of the particles may span from −10% of the mean particle size to ±10% of the mean particle size such as, e.g., ±5% or ±2.5% of the mean particle size. Monodispersity also includes that the particle have the same shape.

In the present disclosure, the term “particle size” (unless otherwise stated) refers to the hydrodynamic or aerodynamic diameter depending on which administration route is considered for delivery of the produced microparticles.

### BRIEF DESCRIPTION OF THE DRAWINGS

The above and other features and advantages of the present disclosure will become readily apparent to those skilled in the art by the following detailed description of exemplary embodiments thereof with reference to the attached drawings, in which:

FIG. 1A illustrates a scanning electron microscopy image of an example mold according to this disclosure,

FIG. 1B illustrates schematic cross section view of the example mold according to this disclosure,

FIG. 2A-2B illustrate a characterization of the mold according to one or more examples of this disclosure,

FIG. 3 illustrates schematics showing different replication scenarios of the mold according to this disclosure,

FIG. 4A-4F illustrate a schematic overview of an example method for producing particles according to this disclosure,

FIG. 5 is a step-by-step illustration containing scanning electron microscopy images associated with the different steps involved in the example method for producing particles according to this disclosure,

FIG. 6A-6D illustrates scanning electron microscopy images of example produced particles according to this disclosure,

FIG. 7 illustrates scanning electron images and in vitro release data pertaining to an experiment with microencapsulated indomethacin according to this disclosure,

FIG. 8 illustrates data pertaining to the fabrication, in vitro and in vivo experiments revolving around microencapsulated delivery of calcitonin,

FIGS. 9A-9C illustrate scanning electron microscopy images showing example produced microparticles after micro-cutting and release onto a compound foil,

FIG. 10 illustrate scanning electron microscopy images showing the microfabricated mold after spray coating a PLGA mold layer onto the mold and then loading the concave compartments with different pharmaceutical compositions, and

FIG. 11 illustrate scanning electron microscopy images showing different particles produced using different mold layers, pharmaceutical compositions, and top layers.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

The figures are schematic and simplified for clarity, and they merely show details which aid understanding the disclosure, while other details have been left out. Throughout, the same reference numerals are used for identical or corresponding parts.

The current disclosure relates to a method for producing microparticles in which a relevant pharmaceutical composition, composed of one or more substances, is encapsulated by one or more biocompatible and preferably biodegradable materials, such as polymer materials. Such materials may include certain enteric materials that are classified as non-biodegradable but are still approved by the Food and Drug Administration (FDA) and/or by the European Medicines Agency (EMA). In the disclosed method, a mold, featuring multiple compartments arranged on a first surface, such as a microstructured first surface, of the mold, is used for producing discrete monodisperse particles consisting of a conformal mold layer, a relevant pharmaceutical composition and a top layer which effectively ensures that the pharmaceutical composition is fully encapsulated. The mold may be microfabricated. The compartments of the mold may be open compartments. Open can herein be seen as being not enclosed, such as having one open side. In one or more examples disclosed herein, the compartments are open towards the first surface of the mold. In one or more example molds, the compartments are close-packed, such as separated from each other by one or more tapered sidewalls featuring edges, wherein each edge has a width of 5 μm or less, such as 1 μm or less. The edges may in one or more examples have a width not exceeding 1 μm. In one or more example molds, the compartments may be concave. The compartments, such as the concave compartments of the mold, may be formed by interconnected positively tapered sidewalls. The sidewalls may be terminated by a sharp apex having a single edge, thereby creating a cutting edge separating each compartment. The mold layer and/or the top layer may comprise multiple materials.

In one or more examples disclosed herein, each compartment has a frustum shape, such as a pyramidal frustum shape. Each compartment may have a base area and a top area. The base area of each compartment may be larger than the top area. The base area, which is outlined by a plurality of interconnected cutting edges, of the compartment may be arranged at the first surface of the mold. By arranging the wider base area of the frustum shape of the compartment at the first surface of the mold, the compartment is wider at the opening arranged at the first surface than at the bottom, such as at the center, of the compartment. Thereby a concave open compartment can be provided. In one or more examples disclosed herein, the base area is 500×500 μm² or less, such as 200×200 μm² or less. In one or more examples disclosed herein, the orthogonal distance from the base area to the top area, such as the distance from the opening of the compartment to the bottom of the compartment, is 500 μm to 10 μm, such as 100 μm to 10 μm.

In one or more examples disclosed herein, a first cross section, such as a cross section in a plane perpendicular to a bottom surface of the compartment, of each compartment has a trapezoid shape. The trapezoid may have a top side, a bottom side and connecting sides that connect the top side with the bottom side. In one or more examples disclosed herein, an angle between the top side and the connecting side is less than 90 degrees.

In one or more example methods, the mold, such as the microfabricated mold, can optionally be subject to deposition of one or more thin (such as having a thickness of 1 μm or less) functional layers. The functional layers may be configured for increasing the hardness and durability of the mold and/or decreasing the surface free energy of the mold to facilitate an easier release of the produced microparticles from the concave compartments after micro-cutting. In one or more example methods, the method comprises one or more of the following steps:
1) Applying a conformal mold layer on the mold, such as on the microfabricated mold, by means of ultrasonic spray coating and/or an analogue method. The mold layer may be applied onto the first surface, such as onto a microstructured first surface, of the mold, such that the open compartments are coated with the mold layer. By coating the open compartments with the mold layer, open shells of the microparticles are provided within the open compartments of the mold. The mold layer may comprise a material, such as a biocompatible and/or biodegradable material, having a first release characteristic, such as one or more of a first water solubility, a first dissolution rate, a first degradation rate and a first permeability of the loaded pharmaceutical composition, at a first condition. In one or more example methods, the first condition corresponds to pH 5-7 and 36-38 degrees C., such as 37 degrees C.
2) Loading the compartments coated with the mold layer with a pharmaceutical composition comprising a drug substance. The pharmaceutical composition may be loaded, and optionally compacted, into the open shells of the microparticles created by the mold layer applied to the first surface of the mold. The shells of the microparticles may remain in the open compartments of the mold.
3) Applying, such as coating, the top layer onto the first surface of the mold, for provision of closed microparticles. The top layer may be applied for example using conventional coating methods including, but not limited to, slot die coating or ultrasonic spray coating. The top layer may comprise a material, such as a biocompatible and/or biodegradable material, having a second release characteristic, such as one or more of a second water solubility, a second dissolution rate, a second degradation rate, and a second permeability of the loaded pharmaceutical composition, at the first condition. In one or more example methods, the second release characteristic may differ from the first release characteristic. In one or more examples disclosed herein, the second release characteristic is the same as the first release characteristic. In other words, the first surface of the mold may be coated with a top layer comprising a biocompatible and/or biodegradable material having a second release characteristic at the first condition for provision of closed microparticles in the multiple individual compartments.
4) Separating the closed microparticles into discrete microparticles by cutting, such as micro-cutting, the mold layer and the top layer using the one or more edges. Thereby, discrete, and optionally monodisperse, microparticles can be provided. After the mold layer and the top layer have been applied the closed microparticles are interconnected by the material of the mold layer and top layer covering the edges separating the compartments. To separate the interconnected closed microparticles from each other, the mold layer and the top layer are cut along the edges separating the compartments. The cutting, such as the micro-cutting, may comprise the edges separating the compartments, such as the sharp cutting edges, on the mold penetrating the mold layer and the top layer, thereby creating discrete particles residing inside the compartments of the mold. A force and/or a pressure may be applied to the mold and/or to the top layer, which cause the edges separating the compartments, such as the sharp cutting edges, to be pushed into and penetrate through the mold layer and the top layer.
5) Optionally, releasing the discrete particles from the mold. Once the closed microparticles have been separated, the individual, such as discrete, particles may be removed from the mold.

### Microfabricated Mold

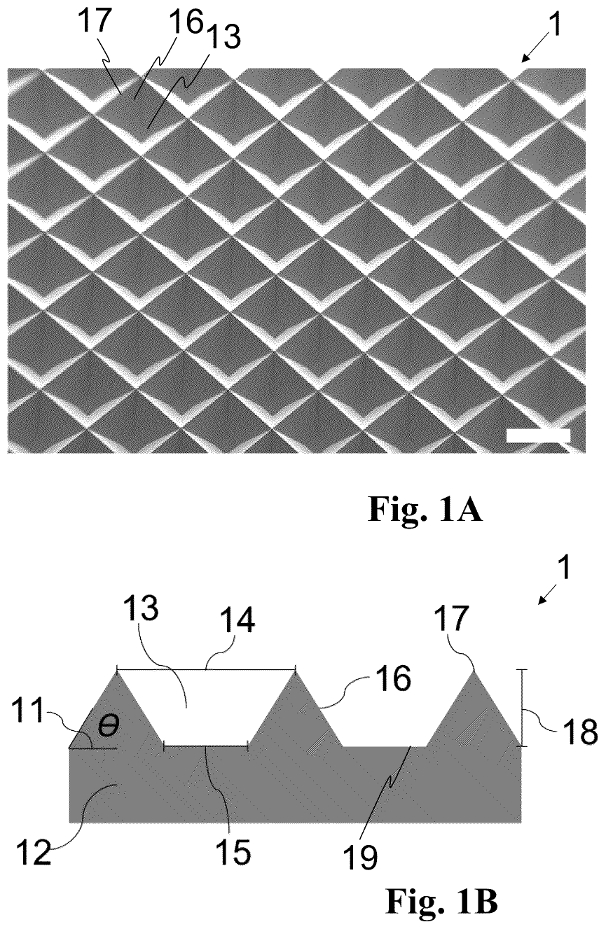

The disclosed method for producing microencapsulated pharmaceutical compositions relies intimately on the topography and material properties of a mold, such as of the microfabricated mold. In one or more examples of the current disclosure, the mold is a planar entity having on the top surface close-packed compartments that are formed by protruding positively tapered sidewalls that are terminated by a sharp cutting edge located at the apex as schematically outlined in FIGS. 1A and 1B. One or more of the interior surfaces of the compartments may be smooth with a surface roughness not exceeding 1 μm. In one or more example molds, all of the interior surfaces are smooth with a surface roughness not exceeding 1 μm. The geometry of the compartments at the top surface (i.e., as outlined by the interconnected cutting edges) may be of a polygonal shape, such as a triangular, a square, a rectangular, a hexagonal shape, or any other shape or combination of shapes, that allow for regular tessellation without overlaps and/or voids forming. In all embodiments of the current invention the taper angle @ of the tapered sidewalls is between 30°-85°, such as 40°-80°, to allow for a conformal deposition of the mold layer and a smooth release of the produced microparticles. Each compartment of the mold may have a frustum shape, such as may be shaped as a regular pyramidal frustum. In other words, the cross section of each concave compartment, such as of the pyramidal frustum, may have a trapezoid shape. The trapezoid may have a top side, a bottom side and connecting sides that connect the top side with the bottom side. In one or more examples disclosed herein, an angle between the top side and the connecting side of the trapezoid is less than 90 degrees. The connecting sides of the trapezoid shape herein correspond to the sidewalls of the compartments. In one or more examples disclosed herein, the surface area of the base surface of the pyramidal frustum, is 500×500 μm² or less, such as 200×200 μm² or less.

The base surface of the pyramidal frustum corresponds to the top side of the trapezoidal cross-section of the compartment. In one or more examples disclosed herein, an orthogonal distance from the top side of the trapezoid to the bottom side of the trapezoid, such as from the base area of the frustum to the top area of the frustum, is in the range of 500 μm to 10 μm, such as in the range of 100 μm to 10 μm. The microparticles obtained, such as obtained using the mold, may thus have a frustum shape, such as a pyramidal frustum shape. In one or more examples, the base area may be rectangular, such as may have two first opposing sides having a first length and two second opposing sides connecting the two first opposing sides, the two second opposing sides having a second length different than the first length. The first length and/or the second length may be in the range of 500 μm to 10 μm, such as in the range of 100 μm to 10 μm. The example geometries of the compartments, allow for producing microparticles that are either pyramids or frustums of the associated polygons outlined by the interconnected cutting edges. The size of such particles can be adequately and accurately defined by the chosen height, taper angle and cross-sectional area of the compartments at the top surface. However, in reality particle ensembles for therapeutic administration may be better described by a mean value of the aerodynamic or hydrodynamic diameter and the associated distribution (or variations in size) as quantified by suitable analytical methods such as Particle Shape Analysis. Other geometries of the compartments are also possible, where the base area may have a circular, triangular hexagonal, octagonal, etc. shape. Independent of the geometry of the compartments, the dimensions of the compartments, such as the dimension of the compartment in the largest direction of extension, may be in the range of 500 μm to 10 μm, such as in the range of 100 μm to 10 μm.

According to one or more examples herein, a mold is disclosed for preparing microparticles for drug delivery, the mold comprising multiple individual open compartments arranged on a first surface of the mold, wherein the open compartments are separated from each other by one or more edges, wherein each edge has a width of 5 μm or less.

In one or more example molds, each compartment has a pyramidal frustum shape.

In one or more example molds, each compartment has a base area and a top area and wherein the base area of each compartment is larger than or equal to the top area.

In one or more example molds, a surface area of the base area is equal to or less than 500×500μm².

In one or more example molds, an orthogonal distance from the base area to the top area of the of each compartment is 500 μm or less.

In one or more example molds, a cross section of each compartment has a trapezoid shape. In one or more example molds, the trapezoid has a top side, a bottom side and connecting sides that connect the top side with the bottom side, wherein the angle between the top side and the connecting side is less than 90 degrees.

In one or more example molds, the mold comprises silicon, such as is made out of silicon.

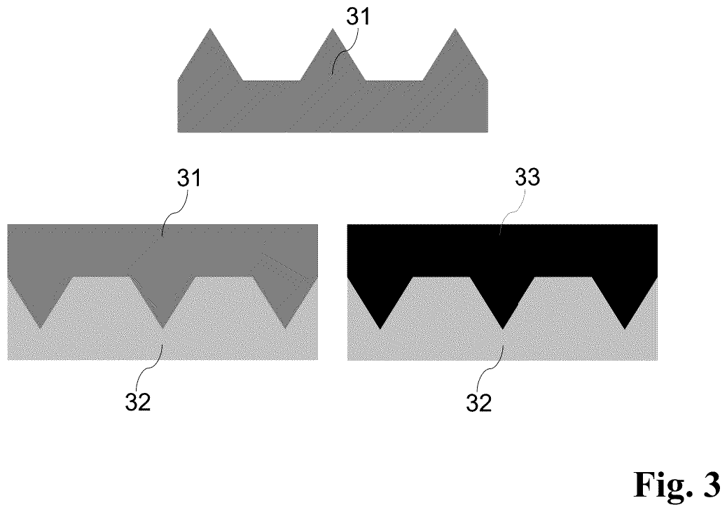

The material of the mold may be selected to provide chemical resistance to solvents used during deposition of the mold layer. Such solvents include water and aqueous solutions as well as polar, polar aprotic and non-polar organic solvents belonging to the conventional categories: aromatic compounds, alcohols, esters, ethers, ketones, amines along with nitrated and halogenated hydrocarbons. More specifically, the material of the mold surface may be selected to withstand repeatable short to medium time (such as up to 1 hour) exposures to organic solvents, such as one or more of ethanol, methanol, acetone, dichloromethane, ethyl acetate, acetonitrile, anisole, isopropanol, propylene glycol methyl ether acetate, chloroform, dimethylformamide, DI water, mild acidic solutions (such as diluted acidic acid), and toluene, without being corroded, dissolved, or otherwise deteriorated to an extent that will impact the topography or surface roughness in a non-negligible manner. The material of the mold surface may be selected to withstand a minimal pH of around 3-4. Effectively, the required chemical resistance limits the number of materials that are suitable for use in the mold. Essentially, most organic materials, including thermoplastic polymers and thermoset polymers as well as epoxies, will be less suited even though epoxies could be feasible for a broad range of solvents. In one or more examples of the current disclosure, the material of the mold may be one or more of organically modified ceramics (such as ORMOCER®), spin-on-glass or other analogues (such as materials that are partially or fully composed of inorganic material that can be cured by UV light or thermally). In one or more examples of the current disclosure, the material of the mold may be a metal, not limited to but including nickel and copper, and alloys. Metals and alloys are generally compatible with organic solvents and bulk workpieces can be micromachined directly, such as using micromilling or laser micromachining, or shims with a suitable thickness (100-3000 μm) can be electroplated on a premade template. Metals and alloys further have the benefit that they are hard and tough materials that will provide a durable mold. Metals and alloys are however prone to oxidation but in the context of the current disclosure, the formation of a thin oxide (thicknesses in the range from 1-200 nm should be acceptable) on exterior surfaces is entirely acceptable. In one or more examples of the current disclosure, the mold is made directly by conventional top-down processing of silicon. This can be done using a combination of thin film deposition, UV photolithography and etching for producing the desired surface topography on a silicon substrate having a thickness in excess of 500 μm. The etching itself may comprise one or more etching steps, such as one or more wet and/or dry etching steps, that allow for tuning of the sidewall taper angle, the surface roughness and/or the explicit local topography associated with the sidewall apex constituting the cutting edge. Using silicon has the benefit that it allows for sculpting the surface topography with great precision. Furthermore, silicon is hard, impurity-free, chemically stable in the presence of aqueous and organic solvents used for dissolving the materials composing the mold layer and top layer and is compatible with the deposition of suitable anti-stick coatings. The fabricated, such as microfabricated, silicon substrate can be used directly as a mold (refer to example 1) or it can be used for pattern replication in other materials that will be used as an intermediate template for production of the final mold. This has been summarized in FIG. 3. Thus, in one or more examples of the current disclosure, the final mold is made by metal or alloy electroplating from an intermediate template and in yet another embodiment of the invention the mold is made by embossing or casting, followed by UV or thermal curing, using the intermediate template, thereby making the final mold in an epoxy, ORMOCER® or spin-on-glass material. It is of course also possible to microfabricate a silicon substrate having the inverse topography of the final mold. The substrate may then be used as a template for electroplating.

In one or more examples of the current disclosure, an inverse template of the mold may be fabricated by 3D-printing. In that case, an intermediate template is not needed, and the metal or alloy mold can be electroplated directly on the 3D printed template. Accordingly, epoxy, ORMOCER® and spin-on-glass molds can also be replicated directly from the microfabricated inverse silicon substrate. The mold materials (such as silicon, epoxies, ORMOCER®s, spin-on-glass, metals, and alloys) are intrinsically hard and durable which supports cutting, such as micro-cutting. However, in one or more examples of the current disclosure, an optional hard coating can be applied to the mold surface having the concave compartments. Such a hard coating may be deposited by one or more of electroless deposition, electroplating, physical vapor deposition or chemical vapor deposition. The material of the hard coating may be one or more of carbides, carbonitrides, nitrides, oxides or borides associated with common metals, such as titanium and/or aluminum, or even diamond like carbon which is commonly used on tools and molds employed for example for injection molding. Thus, in one or more examples of the current disclosure, the mold is coated with a suitable material that increases the surface hardness and durability. To alleviate a smooth release of the microparticles after micro-cutting, the mold surface containing the concave compartments may be provided with an anti-stick coating. The anti-stick coating may be deposited onto the mold. The anti-stick coating decreases the surface free energy associated with the microstructured top surface of the mold, resulting in a reduced adhesion in the interface between the mold and the mold layer. Anti-stick coatings are in general based on silane or fluorinated molecules or polymers that are bound to the surface by physisorption or chemisorption. In the latter case, the antistick coating is covalently bound to the surface which increases the durability of the anti-stick coating. It is paramount that the anti-stick coating will not perturb the topography of the sharp cutting edges or the roughness of the surfaces comprising the concave compartment to any significant extent. Furthermore, the anti-stick coating should not shield the intrinsic hardness of the mold surface, such as of the microfabricated mold surface. Therefore, anti-stick coatings consisting of single molecules deposited by chemical vapor deposition or molecular vapor deposition may be used. Thus, in one or more examples of the current disclosure, the mold surface in contact with the produced microparticles comprises an anti-stick coating. In one or more examples disclosed herein, the antistick coating may comprise chlorosilane chemistries including but not limited to octadecyltrichlorosilane, dichlorodimethylsilane, and perfluorodecyltrichlorosilane. In one or more example methods, the anti-stick coating may be deposited by vapor deposition, such as molecular vapor deposition.

### Mold Layer

In one or more examples of the current disclosure, a mold layer may be deposited on the inner surfaces of the concave compartments of the mold. In one or more example methods, a conformal, and optionally pinhole-free, mold layer is deposited on the inner surfaces of the concave compartments of the mold, such as of the microfabricated mold. Even though a broad range of methods have been developed for depositing thin films on non-planar surfaces having simple or complex topographies, only a few of these methods, such as chemical vapor deposition polymerization, can be implemented for depositing biocompatible and/or biodegradable materials. In one or more examples of the present disclosure, spray coating is used for depositing thin polymer layers directly onto the surface of the concave compartments, such as of the concave compartments, of the mold. Spray coating, more explicitly ultrasonic spray coating, has been used extensively in the semiconductor industry for coating surface topographies with photosensitive polymer layers in the production of e.g., microelectromechanical systems. Furthermore, ultrasonic spray coating is gaining momentum in the production of novel photovoltaic devices and for coating stencils and implants with polymer layers ranging in thickness from a few nanometers up to 100 μm or more. In one or more examples of the current disclosure, spray coating (such as electro spray coating, air-brush spray coating, and/or ultrasonic spray coating) is used for depositing a thin layer, having an overall thickness of 1-100 μm, such as 1-50 μm, on the interior surfaces, such as on the side walls and bottom surface, of the compartments that are located on the top surface of the mold, such as of the microstructured mold. The mold layer may comprise one or more sublayers that are coated in a sequential manner. To increase the adhesion between sublayers and the overall quality of individual sublayers, the surfaces of the sublayers may be functionalized using dry oxidation methods, such as corona discharge treatment, atmospheric or low pressure oxygen plasma treatment or photooxidation, to create functional groups, such as hydroxyl groups, in the interface between adjacent sublayers. In one or more examples of the current disclosure, one or more sublayers may be composed of one or more materials. The one or more materials may have been dissolved and mixed in an appropriate solvent or combination of solvents prior to spray coating. In one or more embodiments of the current disclosure, a dynamic viscosity of the solutions used for spray coating the individual sublayers is in the range of 0.1-500 cP (1 cP=1 mPa·s), such as in the range of 0.1-50 cP. According to one or more examples of the current disclosure, the material of the mold layer may be one or more of polyurethane (PUR), polycaprolactone (PCL), polylactic acid (PLA) in all stereoisomeric forms, polyglycolic acid (PGA), co-polymers of PLA and PGA known as poly lactic-co-glycolic acid (PLGA), polyvinylpyrrolidone (PVP), polyvinyl alcohol (PVA), polyethylene glycol (PEG), polyacrylic acid (PAA), hydroxypropyl methylcellulose (HPMC), and any other polymer material that is biocompatible and/or biodegradable. In one or more example methods, the material of the mold layer is PLGA 50:50 (Mw=38-54 kDa) dissolved in ethyl acetate to obtain a 1 wt % solution used for spray coating. In one or more example methods the mold layer comprises two sublayers where the first layer is PCL (Mw=80 kDa) dissolved in dichloromethane to obtain a 0.5 wt % solution for spray coating and the second layer is PLGA 50:50 (Mw=38-54 kDa) dissolved in ethyl acetate to obtain a 1 wt % solution for spray coating. In one or more examples of the current disclosure, the mold layer comprises a biocompatible and/or biodegradable material, such as a polymer, having a first release characteristic at a first pH value, such as at pH 5-7, and a first temperature, such as at 36-38 degrees C, such as at 37 degrees C. In one or more examples of the current disclosure, one or more of the materials may be common synthetic enteric materials, where an increased solubility is triggered in response to the local pH or enzyme environment, such as one or more of methyl acrylate-methacrylic acid copolymers (such as Eudragit®), cellulose acetate phthalate (CAP), hydroxyl propyl methyl cellulose phthalate (HPMCP), hydroxyl propyl methyl cellulose acetate succinate (HPMCAS), and polyvinyl acetate phthalate (PVAP). In one or more examples of the current disclosure, the material may be derived from naturally occurring polymers, such as polysaccharides including alginate, carrageenan, chitin, chitosan, cellulose, dextran, starch, hyaluronic acid, and pullulan. The mold layer constitutes a main part of a solid-state barrier that is encapsulating the pharmaceutical composition. Said barrier may be fully impermeable with regards to the pharmaceutical composition, or it may have an engineered permeability governed by the materials of the mold layer. In one or more examples of the current disclosure, the mold layer is impermeable to the pharmaceutical composition and the release is solely mediated by diffusion through or dissolution/degradation of the top layer. In one or more examples of the current disclosure, the mold layer is a permeable membrane through which the active ingredients in the pharmaceutical composition can diffuse thereby alleviating a tailored release profile. In one or more examples of the current disclosure, the mold layer is composed of a material, such as an enteric or water soluble material, that can dissolve quickly, so that a release of the pharmaceutical composition happens in an instant manner in response to a local chemical and/or enzymatic environment. In the spray coating process, one or more nozzles, such as an array of nozzles, may be used for effectively covering the entire surface area of the mold. In one or more example methods, the deposition of the mold layer may be performed by moving the nozzle(s) across the mold surface in a plane which is parallel with the mold surface or conversely by moving the mold itself in relation to one or more static nozzles. The mold layer deposition process may comprise one or more cycles (such as sub-coating steps) to achieve the desired mold layer thickness and composition. In one or more example methods, both the mold layer and the top layer are impermeable, and a release of the pharmaceutical composition is mediated by degradation (such as by a potentially slow erosion) of the materials of the mold layer and/or the top layer.

### Pharmaceutical Composition

The actual substance conveying the physiological effects associated with the produced microparticles is the pharmaceutical composition. In one or more examples of the current disclosure, the pharmaceutical composition may comprise solid or semi-solid state materials, such as preprocessed powders, microparticles, nanoparticles, slurries, pastes and analogue compositions that can be loaded inside the compartments of the mold. One or more example pharmaceutical compositions according to the current disclosure are provided in Example 7 below and also in examples 3-6. When considering the microscale morphology of the pharmaceutical composition, it must be demanded that the grain size, particle size or any other relevant measurand used to describe the spatial size of the constituents comprising the composition is kept small as to allow for efficient loading inside the microscopic concave compartments. In reality, the composition will be composed of entities that are seldomly monodisperse and in that case, it will be assumed that the ensemble can be described adequately by a quantifiable size distribution. In one or more examples of the current disclosure, the largest constituent of the pharmaceutical compositions has an average size not larger than 6-25%, such as not larger than 10-20%, of the inner volume of the compartments of the mold, such as of the microfabricated mold. Therefore, in one or more example pharmaceutical compositions the constituents of the pharmaceutical composition may be preprocessed via one or more preprocessing steps, comprising one or more of milling, grinding, spray drying, freeze drying, rapid expansion of supercritical solutions, supercritical anti-solvent techniques, particles from gas saturated solutions and other micronization methods. The pharmaceutical composition may comprise one or more drugs, including, but not limited to, peptide drugs, protein drugs, probiotics, macromolecules, or vaccines. In addition to the active pharmaceutical ingredient, the pharmaceutical composition may comprise one or more excipients that aim at i) increasing the efficacy/potency of the active ingredient, ii) enhancing the systemic uptake of the active ingredient, iii) stabilizing the active ingredient, and/or iv) facilitating production and stability of the produced microparticles. Such excipients include, but are not limited to, adjuvants, permeation enhancers, antiadherents, glidants, disintegrants, lubricants, diluents, fillers, and agglutinants. Adding the excipients ensures that the maximum efficacy/potency of the active pharmaceutical ingredient is conveyed but also that an efficient loading and compaction of the pharmaceutical composition into the concave compartments of the mold, such as the microfabricated mold, is enabled. In the current disclosure, the pharmaceutical composition may be dosed on the top surface of the mold and effective loading and/or compaction may be achieved mechanically by moving for example an antistatic brush, a spatula, and/or a squeegee (or similar) in a direction that is parallel with the top surface of the mold. In one or more example methods, the compaction may be performed by pressing a soft and flexible pad against the top surface, and/or by having vibration-assisted compaction and/or distribution. This ensures that the composition is loaded inside the mold-layer-coated concave compartments. In one or more example methods, static electricity on the top surface of the mold layer may be eliminated prior to loading of the pharmaceutical composition to facilitate an easier loading of the composition. In one or more example methods, functional groups, such as hydroxyl groups, may be promoted on the top surface of the mold layer to increase adhesion to the subsequently deposited top layer.

### Top Layer

Following the loading of the pharmaceutical composition into the compartments of the mold, a suitable top layer is deposited on the mold. In one or more examples herein, the top layer is pinhole-free. At this stage, the pharmaceutical composition is loaded inside the mold-layer-coated compartments of the mold. The top layer can be composed of one or more sublayers, each consisting of one or more materials. To increase the adhesion between the one or more sublayers and the overall quality of the individual sublayers, the surfaces of the sublayers may be functionalized using dry oxidation methods, such as corona discharge treatment, atmospheric or low pressure oxygen plasma treatment or photooxidation, to create functional groups, such as hydroxyl groups, in the interface between adjacent sublayers. Such surface functionalization methods can also improve adhesion between the top surface of the mold layer and the first sublayer constituting the top layer. In the top layer deposition process an essentially flat surface is to be coated. The uniform coating of such a surface can be performed by numerous coating methods, such as spray coating, slot die coating, roll coating, knife blade coating, and/or spin coating. In one or more examples of the current disclosure, the top layer is deposited using a non-contact coating method, such as slot die coating or spray coating, in which there is no direct contact between a coating tool, such as an ultrasonic nozzle or a slot die head, and the mold surface. During the top layer deposition, the coating tool, such as the slot die head, or one or more spray nozzles, may be translated across the mold in a plane that is parallel to the upper mold surface. Conversely, the mold can be translated in relation to a static slot die head or one or more static nozzles that allow for complete coverage of the entire mold surface area. In one or more example methods disclosed herein, the top layer deposition may comprise one or more cycles (such as sub-coating steps) in order to achieve a desired top layer thickness and/or composition. In one or more examples of the current disclosure, the top layer thickness is in the range of 1-100 μm, such as 1-50 μm, such as 1-20 μm. The materials comprising the top layer can be one or more of the materials used for the mold layer and mentioned in the section “Mold layer”. In one or more examples disclosed herein, the top layer comprises a material having a second release characteristic, such as a second water solubility, a second dissolution rate, a second degradation rate, and/or a second permeability of the loaded pharmaceutical composition at the first pH value, such as pH 5-7, and at a first temperature, such as at 36-38 degree C., such as 37 degrees C. In one or more examples herein, the second release characteristic is at least 10 times greater than the first release characteristic. In one or more examples herein, the second release characteristic is the same as the first release characteristic. In one or more examples of the current disclosure, liquid and/or solid plasticizers can be added to the material or materials comprising the top layer to modify the mechanical properties of the top layer. Examples of plasticizers include glycerin, polyethylene glycol, propylene glycol, sorbitol sorbitan solutions, triacetin, dibutyl sebacate, castor oil, polyethylene glycol monomethyl ether, acetyl triethyl or tributyl citrate, triethyl or tributyl citrate. By adding liquid and/or solid plasticizers to the top layer, a brittleness of the materials used in the top layer can be reduced, which reduces the risk of the top layer fracturing due to shearing in the micro-cutting process. In one or more examples of the current disclosure, a material of a layer, such as the material of the top layer and/or the mold layer, comprises a film-forming polymer selected from a group consisting of co-polymers based on polymethacrylic acid and methacrylates, ethyl acrylate and methyl acrylate, co-polymers of acrylic and methacrylic acid esters, hydroxypropyl methylcellulose phthalate, cellulose acetate phthalate, polyvinyl acetate phthalate or mixtures thereof. In one or more example methods, the top layer may comprise any of the materials as specified for the mold layer. The material of the top layer may be the same or may be different than the material for the mold layer. In one or more examples of the current disclosure, the material of the top layer comprises a film-forming polymer selected from the group consisting of cellulose, cellulose derivatives, methylcellulose, hydroxypropylcellulose, hydroxypropyl methylcellulose, and mixtures thereof. In one or more examples of the current disclosure, the material of the top layer comprises a plasticizer, such as a polyethylene glycol. In one or more examples of the current disclosure, the plasticizer is liquid. When using liquid plasticizers, it is paramount to ensure that evaporation and or diffusion of the plasticizer is minimized prior to the micro-cutting and release steps. In the current disclosure, a brittle material can be seen as a material that cracks or ruptures at moderate levels of strain, such as at 1-5% strain, and may require liquid and/or solid plasticizers to be used to prevent cracking and/or rupture of the material. On the other hand, top layers comprising materials that can sustain elastic or plastic deformations ranging from 5-50% will be considered feasible for microparticle production. In a final note, top layers comprising highly flexible materials that support elastic and/or plastic deformations of more than 50% without reaching the fracture point will be considered less feasible and problematic as the inclusion of such materials could result in distortion of the overall microparticle morphology and ultimately prevent fabrication of discrete microparticles using micro-cutting. In other words, the material of the top layer can be a material where the elongation at break is between 5-50%. The top layer deposition may in one or more examples be done by coating one or more sublayers in a sequential manner. Each sublayer may comprise one or more materials that are dissolved in an appropriate solvent or combination of solvents. In one or more examples of the current disclosure a dynamic viscosity of the solutions used for the different layers is between 0.1-100 cP when spray coating is employed or between 100-5.000 cP when slot die coating is employed. To ensure tightly sealed microparticles, the adhesion between the top layer (or more precisely the first sublayer or the top layer if said layer comprises multiple layers) and the pharmaceutical composition loaded inside the mold-layer-coated compartments must be considered. As the top layer directly interfaces the top surface of the mold layer as well as the pharmaceutical composition it will be highly beneficial to use a solution consisting of one or more materials dissolved in a solvent (or combination of solvents) which is a poor solvent for the pharmaceutical composition but a good solvent for the material or materials comprising the top surface of the mold layer. In this case, a tight seal can be obtained along the periphery of the produced microparticles which prevents premature leakage of the encapsulated pharmaceutical composition, while also strengthening the bulk mechanical properties of the microparticles. The top layer may constitute a second part of the solid-state barrier encapsulating the pharmaceutical composition. Depending on the administration route and the desired modus operandi during release, the top layer may constitute an impermeable barrier and release is solely mediated by diffusion through and/or dissolution of the mold layer. In one or more examples herein, both the mold layer and top layer are impermeable and not readily soluble, and release is only mediated by slow degradation of the solid-state barriers. This could be feasible for administration of depot pharmaceutical compositions requiring a delayed release. The top layer may, in one or more examples, also constitute a permeable barrier through which the loaded pharmaceutical composition can diffuse, thereby establishing an extended release. In one or more examples of the current disclosure, the top layer can be composed of one or more materials that can dissolve quickly, so that the release of the pharmaceutical composition happens in an instant manner in response to the local chemical and/or enzymatic environment. Such materials include for example, enteric materials where the dissolution is triggered by changes in pH value. In other words, in one or more examples herein, the top layer is composed of one or more enteric materials.

### Micro-Cutting

The process in which discrete particles are obtained is interchangeably called cutting or micro-cutting in the current disclosure. Cutting is similar to conventional mechanical punching in which a matching die and punch set is used to produce e.g., metal or rubber components in an industrial setting. In the disclosed method for producing microencapsulated pharmaceutical compositions, cutting may be performed by applying a planar parallel force, such as a mechanical, pneumatic, or hydraulic force, or pressure on the microfabricated mold. Prior to cutting, the mold has been subject to mold layer deposition, loading of the pharmaceutical composition and finally top layer deposition. In the cutting process, the sharp cutting edges associated with the sidewalls forming the close-packed concave compartments, may be forced through the mold layer and top layer. The thickness of the mold layer residing on the top of the cutting edges is negligible, so the main task is to cut through the top layer. Micro-cutting is different from mechanical punching as no counter die is used. Instead, a plate, foil, or film of backing material can be situated in direct contact with the top layer. It is paramount that the hardness of the backing material is lower than the hardness associated with the top surface of the microfabricated mold, as the edges separating the individual compartments, such as the cutting edges, must penetrate into the backing material. The edges separating the individual compartments, such as the cutting edges, may have a width of 5 μm or less, such as 1 μm or less. If a wider edge was used, the mold layer would most likely also be deposited on a planar segment of the cutting edge, which would reduce the ability of the cutting edge to cut through the mold layer and/or the top layer to cut the particles. As the cutting edges sink into the backing material, the mold layer and the top layer is sheared, stretched, and ultimately cut as the maximum fracture strain of the layer is reached. The maximum fracture strain is dictated by the mechanical properties of the materials comprising the mold layer and top layer. In that regard, cutting is obtained when the force, such as the mechanical, pneumatic, or hydraulic force, or pressure applied in the process is sufficient for promoting a situation in which the maximum fracture strain is accomplished along all interconnected cutting edges of the mold. In one or more example methods a cutting force of 500-5000 N/cm², such as 1000-3000 N/cm² has been used. Once the mold layer and the top layer have been cut, discrete microparticles are produced and said particles will reside inside the concave compartments of the mold after the cutting process. The cutting process may be executed at room temperature and results in a compaction of the loaded pharmaceutical composition. The force or pressure needed to obtain efficient cutting may be dictated by i) the materials comprising the mold layer and top layer, ii) the exact topography and mechanical properties of the top surface of the mold, such as the microfabricated mold, and iii) the mechanical properties of the backing material which effectively serves as the die in the micro-cutting process. In general, micro-cutting is less compatible with the use of brittle materials in the mold layer and top layer. As the mold layer, which is in intimate contact with the surfaces, such as the concave surfaces of the microfabricated mold, is supported by the mold during cutting a certain degree of brittleness is acceptable when it comes to the materials comprising the mold layer. Conversely, having brittle materials in the top layer is detrimental in terms of realizing a high yield in the cutting process: as the cutting edges sink into the backing material the top layer will be subject to deformation leading to a slightly concave morphology of the top layer after the cutting process. The degree of deformation can be determined by the maximum fracture strain and the mechanical properties of the top layer and the backing material. According to the current disclosure, it is considered beneficial to utilize a backing material where i) the hardness is comparable to or larger than the maximum hardness associated with the materials comprising the mold layer and top layer, and optionally ii) the intrinsic properties allow for substantial deformations sufficient for realizing a scenario in which the maximum fracture strain promoted at the interconnected cutting edges is not resulting in massive plastic deformations or even rupture or cutting of the backing material. Suitable materials to be used as backing materials include, but are not limited to, hard elastomers, such as natural or synthetic rubbers, and thermoplastic elastomers having a Shore D hardness in the range 0-100, such as 50-100, and flexible polymers and compound materials having a Shore D hardness exceeding 100. In one or more examples disclosed herein, intermediate hardness metal foil or film with a Brinell hardness in the range HBN=10-200, could be used, provided that the hardness is maintained below the hardness associated with the top surface of the mold, such as the microfabricated mold. In this case, the backing foil will be subject to plastic deformations as the cutting edges penetrate into the foil or film. The cutting process, such as the micro-cutting process, may have a short cycle time, such as a cycle time not exceeding 60 seconds, such as in the range of 1-30 seconds, such as in the range of 1-10 seconds. The cutting process time may be mainly determined by the magnitude of the cutting force or pressure and the intrinsic properties of the materials comprising the mold layer, the top layer, and/or the backing material. Optionally, the cutting process can be adapted for use on a machine that allows for ultrasonic application of a force or a pressure for facilitating both cutting as well as the subsequent release of the produced microparticles.

### Particle Release

After the cutting process, the discrete microparticles are located inside the concave compartments of the mold, such as the microfabricated mold. In the current disclosure a microfabricated mold which can feature an anti-stick coating or layer has been described. By using an anti-stick layer, which may have a low surface free energy on the surfaces, such as on the concave surfaces, of the mold the adhesion in the interface between the smooth concave surfaces of the mold and the mold layer is kept minimal. In the current disclosure, surface free energy, surface energy and interfacial free energy may be used interchangeably. It is expected that the adhesion, as measured e.g., in a direction perpendicular to the mold top surface, depends on the material directly in contact with the mold surface. Polymer materials like e.g., polyvinyl alcohols and most enteric materials, such as Eudragit®, are intrinsically hydrophilic and possess a high surface free energy. Such materials are expected to exhibit an increased adhesion to the surfaces of the mold. Conversely, hydrophobic polymer materials, such as polycaprolactone, that exhibit low intrinsic surface free energy, are expected to have poor adhesion to the concave mold surfaces. Another aspect that may impact the adhesion between the produced microparticles and the mold is the surface roughness associated with the compartments of the mold. Having a high surface roughness can lead to increased adhesion due to e.g., mechanical interlocking of the material in the mold layer that is directly interfacing the compartments of the mold. Clearly, this is not beneficial in terms of a smooth release of the produced particles, which underpins the requirement of a low surface roughness as stated in the section “Microfabricated mold”. When considering feasible methods for particle release, there is a distinction between non-contact based and contact based methods. Non-contact based methods include vacuum suction, ultrasonic vibration, such as intermediate to high power ultrasonic vibration, and analogue methods that produce either a static or temporally fluctuating force acting in a direction perpendicular to the top surface of the mold. The force must be sufficient for overcoming the adhesive strength associated with the interface between the mold and the mold layer. The adhesive strength may be significantly lower than 100 kPa (i. e, 0.1N/mm² ), such as 0.001-1 kPa or lower. Contact based release methods revolve around utilizing a flexible release foil (also referred to as carrier foil in the current disclosure) having a bulk Young's modulus of E=0.1-100 MPa and/or a Shore A surface hardness ranging from 0-100, such as 50-100, which is optionally capable of sustaining elastic deformations ranging from 1-50%. The use of a soft and flexible carrier foil allows for obtaining a conformal and intimate contact between the top surface of the carrier foil and the top surface of the top layer using low to intermediate pressures/forces in the release process. In one or more examples disclosed herein, the carrier foil may comprise one or more of i) a single material exhibiting high surface free energy, ii) a single material which has been surface activated in a wet or dry process that generates functional groups, such as hydroxyl groups, on the top surface of the foil, and iii) multiple materials of which the material on the top surface acts as a temporary adhesive where the adhesive strength is governed by van der Walls or electrostatic forces. In the latter case, the carrier foil is a compound foil consisting of a soft and flexible polymer with a thin layer of adhesive, such as residue-free adhesive, on the top surface. In one or more example methods in the current application a 65 μm thick plasticized polyvinylchloride (PVC) foil (E=35 MPa) with a 10 μm thick layer of pressure sensitive acrylic-based adhesive is used as the carrier foil. When considering contact based release methods, the adhesion between the carrier foil and the top surface of the top layer should be selected so that it exceeds the adhesion between the mold layer and the mold. In addition, it may also be required that the adhesion between the mold layer and top layer exceeds the adhesion between the mold layer and the mold and ideally the adhesion between the top layer and the carrier foil used for release. Fulfilling all these requirements enables a smooth release of the micro-cut particles from the compartments, such as the concave compartments, of the mold, and a seemingly effortless removal/collection of the released particles after the release cycle. During the release cycle, the carrier foil may be brought into contact with the top surface of the microfabricated mold. Subsequently a mechanical, pneumatic, or hydraulic force or pressure may be applied to the back side of the carrier foil, thereby promoting an intimate contact between the top surface of the carrier foil and a back side (such as the top surface of the top layer) of the cut, such as micro-cut, particles. In one or more example methods, a planar parallel force of 1-1000 N/cm², such as 1-500 N/cm² has been applied during microparticle release. In one or more examples disclosed herein, the mold, having in the compartments the micro-cut particles, can be pressed against the carrier foil during release. The release may be carried out at room temperature in a short cycle time process, such as a process where the cycle time is not exceeding 60 seconds. Afterwards, the carrier foil may be released in a direction perpendicular to the mold surface as to ensure minimal friction between the produced microparticles and the compartments of the mold. Following the release cycle, the particles can be collected from the carrier foil and either loaded directly into a suitable dosage unit, such as a capsule, or stored in a macroscale container for future use.

### Particle Sizes and Administration Routes

The proposed fabrication method is intrinsically generic when it comes to the produced particle sizes and allows for producing bulk amounts of microparticles, such as monodisperse microparticles, targeting different administration routes. The size and shape of the produced microparticles is largely dictated by the topography of the mold. Said mold may be produced to have compartments, such as concave compartments, where the size of the compartment ranges from a few hundred nanometers and up into the millimeter or even centimeter range. In the latter case, the distinction between particles produced using the disclosed method and conventional dosage forms such as capsules or tablets is of course not overly obvious. When considering administration of microencapsulated pharmaceutical compositions there is a vast range of administration routes that can be considered. Such routes include oral, buccal, vaginal, rectal, and ocular routes, where there may be no certain requirements to the size, shape and monodispersity of the microparticles. However, other administration routes such as parenteral injections (such as intramuscular, subcutaneous, intravenous, and intrathecal injections) and perhaps more relevant nasal and pulmonary routes pose certain strict requirements on the size, shape and monodispersity of the microencapsulated pharmaceutical composition. In general, particles having a size of 30-120 μm are considered feasible for nasal delivery, whereas particles for pulmonary delivery are characterized by sizes in the range of 1-20 μm. The disclosed method can be used for producing particles for all the mentioned administration routes, including the nasal and pulmonary route. The main obstacles to be overcome when implementing the disclosed method for producing very small particles (such as particles having sizes in the range of 1-20 μm) is associated with the deposition of the mold layer and the preprocessing steps needed for making a pharmaceutical composition in which the maximum size of the constituents comprising the composition is kept sufficiently small. As the particle size approaches 1 μm it is of course also debatable whether or not there would be a clinical and/or commercial incentive for producing engineered microparticles having a relatively low loading capacity. From a production point of view, depositing a conformal and pinhole free mold layer with a sub-1 μm thickness is definitely entirely realistic, even in the case of depositing the mold layer on an anti-stick coated microfabricated mold. The most prominent barrier is thus related to the formulation of a suitable pharmaceutical composition which allows for loading inside the compartments, such as the concave compartments, of the mold. Therefore, in one or more examples disclosed herein, the fabrication method is aimed at producing microparticles with sizes ranging from 1 to 1000 μm, such as 10-500 μm, such as 10 μm to 20 μm. In one or more examples disclosed herein, the microparticles, such as the microparticles obtained using the mold, may have a pyramidal shape and/or a frustum shape, such as a pyramidal frustum shape, such as a truncated pyramidal shape. The sizes mentioned herein may be the size of the particle in the largest direction of extension.

### Example Implementation of the Fabrication Method

In the following, an example process flow for producing a microencapsulated pharmaceutical composition using the disclosed fabrication method is described. Prior to initiating the process flow, a mold, such as a microfabricated mold having an anti-stick coating may be provided. The produced particles may comprise a semi-permeable mold layer, a peptide drug, and an enteric top layer. Such particles may be aimed at triggered release in the small intestine after oral administration:
1) The mold may be cleaned to remove particulate and lipid contamination. This may be done by flushing the mold with alcohol, such as ultra-pure isopropyl alcohol, and subsequent drying, such as in a stream of purified nitrogen.
2) Low wt % solutions of PCL in dichloromethane solvent and PLGA in ethyl acetate solvent may be prepared by dissolving the polymers in the solvents.
3) The mold may be placed inside a spray coater and a suitable ultrasonic nozzle or array of nozzles may be mounted to the spray coater, such as mounted inside the spray coater. The mold may be heated to 40° C. using a hot plate prior to initiating a mold layer deposition. The hot plate may be comprised in the spray coater.
4) The first conformal mold layer, which may constitute a 1-5 μm thick layer, such as a PCL layer, that facilitates microparticle release, may be deposited by ultrasonic spray coating. The coated mold may be left on the hot plate for total evaporation of the volatile dichloromethane.
5) In one or more example process flows, the PCL sublayer may be subject to a dry oxidation process that promotes functional groups, such as hydroxyl groups, on the surface of the PCL sublayer prior to coating a second sub-layer. This step is aimed at increasing the adhesion between the sublayers composed of dissimilar polymer materials.
6) The second sublayer, which may consist of PLGA dissolved in ethyl acetate, may be deposited by ultrasonic spray coating until a final mold layer thickness of 10 μm is reached. Again, remaining solvent is evaporated by leaving the mold on the hot plate.
7) In one or more example process flows, the PLGA sublayer may be subject to a dry oxidation process that promotes functional groups, such as hydroxyl groups, on the surface prior to drug loading and top layer coating. This step is aimed at increasing the adhesion between top surface of the mold layer and the top layer.
8) The pharmaceutical composition, such as a peptide drug mixed with a permeation enhancer, may be preprocessed by grinding, to provide a powder having a maximum grain size not exceeding 5 μm.
9) The pharmaceutical composition may be deposited on the top surface of the mold and the powder may be mechanically spread over the mold to achieve loading of the compartments, such as the concave compartments.
10) A low wt % solution of a suitable enteric polymer (e.g., Eudragit® L100) dissolved in a relevant solvent may be prepared by mechanical mixing. To ensure that the enteric top layer is flexible, a suitable solid plasticizer (e.g., PEG 6000) may also be added to the solution.
11) The drug loaded mold may be situated in the ultrasonic spray coater. The hot plate is kept at 40° C. The top layer is deposited by ultrasonic spray coating and the process is terminated when the top layer has reached a thickness of approximately 5-10 μm. The mold is left on the hotplate until the solvent has been evaporated.
12) The mold may be placed in a parallel plate press with the top side in direct contact with the backing material used for cutting, such as micro-cutting. The cutting may then be carried out at a pressure in the range from 1000-3000 N/cm² for 30-60 s.
13) The particles are subsequently released from the mold. The particles may be released onto a thin flexible carrier foil with a residue free pressure sensitive adhesive using a pressure in the range from 50-500 N/cm² for a duration of 30-60 s.
14) Following the release, the particles may be collected from the carrier foil and may be loaded into a suitable capsule for oral dosing.

The example above is to illustrate what can be conceived as a typical process flow for producing microparticles.

FIG. 1A shows a scanning electron microscopy image of an example mold 1, such as the microfabricated mold, according to one or more examples of the current disclosure. The scanning electron microscopy image is acquired at a tilt angle of 30°. From the image, which shows a top surface of the mold 1, it can be seen that the mold 1 comprises a plurality of compartments 13, such as a plurality of concave compartments. The plurality of compartments 13 may, in the example shown in FIG. 1A, be shaped as regular pyramidal frustums, such as having a plurality of sloped sidewalls 16 each having a side wall taper angle ⊖. The plurality of compartments may be close-packed according to the definition provided in the definition section. The individual compartments 13 are separated by positively tapered sidewalls that are terminated at the top surface by a sharp apex which effectively constitutes an edge 17 separating the openings of the individual compartments. Each edge 17 may have a width of 5 μm or less, such as 1 μm or less. The edge 17 may herein also be referred to as a cutting edge, that may be used for cutting through the mold layer and the top layer for separating the microparticles from each other. The scale bar corresponds to 200 μm.

FIG. 1B discloses a schematic cross section view of the example mold 1 along a first plane. The schematic highlights features of the mold 1. The mold 1 comprises a bulk unstructured part 12, and one or more concave compartments 13 residing on a top surface of the mold 1, such as on a top surface of the bulk unstructured part 12. The plurality of compartments 13 may have a pyramidal frustum shape, which is formed by a plurality of sidewalls 16. The sidewalls 16 may be sloped and may each have a side wall taper angle ⊖11 to a bottom surface 19 of the compartment. In other words, each compartment of the mold 1 may be shaped as a regular pyramidal frustum. In other words, the cross section of each concave compartment 13, such as of the pyramidal frustum, may have a trapezoid shape. The trapezoid has a top side, a bottom side and connecting sides that connect the top side with the bottom side. In one or more examples disclosed herein, an angle between the top side and the connecting side of the trapezoid is less than 90 degrees. The connecting sides of the trapezoid shape herein correspond to the sidewalls 16 of the compartments. In one or more examples disclosed herein, the surface area of the top side of the trapezoid, such as a top surface of the pyramidal frustum, is 500×500 μm² or less, such as 200×200 μm² or less. In other words, the distance between two parallel cutting edges may in one or more examples disclosed herein be 200 μm. In one or more examples disclosed herein, an orthogonal distance from the top side of the trapezoid to the bottom side of the trapezoid is in the range of 500 μm to 10 μm, such as in the range of 100 μm to 10 μm. The microparticles obtained, such as obtained using the mold, may thus have pyramidal shape. In one or more examples herein, the trapezoid shape is an isosceles trapezoid shape. In other words, the side wall taper angles 11 of each side wall of the compartment and a length of each of the side walls 16 have equal measures. In other words, all side wall taper angles 11 of the compartment is the same, and the length of all side walls 16 of the compartment is the same. The sloped sidewalls 16 are terminated by sharp cutting edges 17. The concave compartments 13 have a first distance 14, such as a length, between neighboring cutting edges 17, a width 15 of the bottom part of the concave compartment 13 and a depth 18 of the concave compartments 13 as measured from a top apex constituting the cutting edge 17 to the bottom of the concave compartments 13. In the example mold 1 shown in FIG. 1A and B, the mold 1 may have the same cross section along a second plane orthogonal to the first plane.

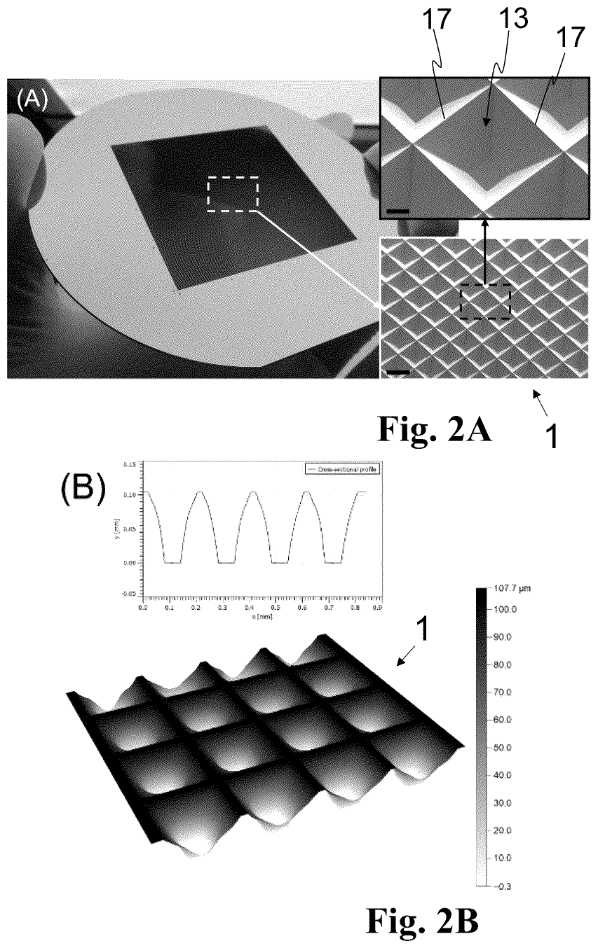

FIGS. 2A and 2B show a characterization of the mold, such as the microfabricated mold, according to one or more examples disclosed herein. FIG. 2A is a picture of the final mold 1, such as a final silicon mold. The example substrate of the mold has a diameter of 100 mm and a center area of 50×50 mm² contains a 2D array of 250×250 compartments that allows for producing up to 62500 discrete particles. The insets of FIG. 2A show scanning electron microscopy images acquired at the tilt angle of 30°. In the lower insert, the close-packed configuration of the concave compartments is clearly evident. The scale bar corresponds to 200 μm. The upper insert shows a high-magnification image of a single compartment 13 and the associated sharp cutting edges 17. The scale bar in the upper insert corresponds to 40 μm. FIG. 2B shows topography data from vertical scanning interferometry measurements on the mold 1 prior to a final dry etch used for sculpting the cutting edges 17. The image shows a 3D topography associated with the concave compartments 13 and the inset shows a cross-sectional profile in which the average compartment depth is 103 μm. From the cross-sectional profile, an example sidewall taper angle of approximately 55° can be estimated.

FIG. 3 are schematics showing different replication scenarios when starting out with a prefabricated template 31 having the desired topography, such as the topography of the mold 1 disclosed in relation to FIGS. 1A and 1B. The template may be used as a mold 1 but it can also be used for producing an inverse replication 32 of the mold 1. Said replication can then be used to make an additional inverse replication 33 on which the surface topography is approximately identical to the topography on the original prefabricated template 31.

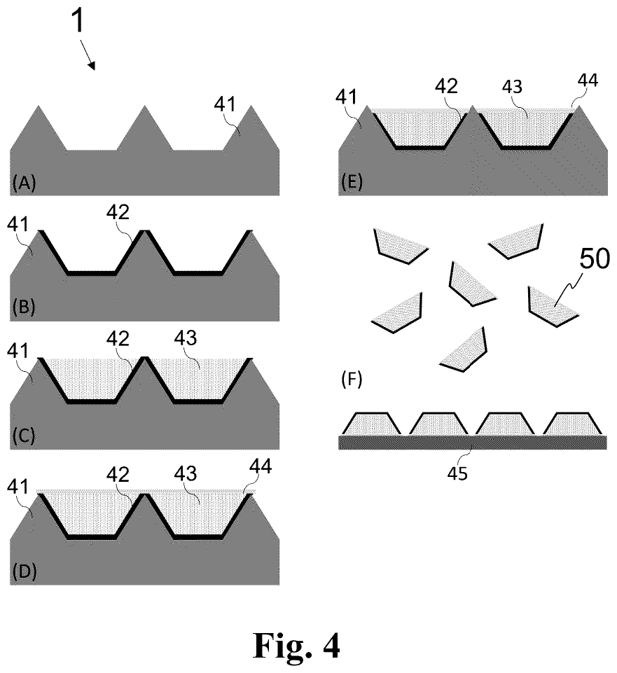

FIGS. 4A-4F show a schematic overview of the method, such as the process flow, for producing particles according to the current disclosure. As shown in FIG. 4A, initially a mold 1, having on the top surface thereof a topography where close-packed concave compartments 13 are separated by sharp cutting edges 17, is provided, for example by producing the mold using conventional microfabrication methods, such as the replication method shown in relation to FIG. 3. The mold 1 may correspond to mold 1 disclosed in relation to FIGS. 1A and 1B. In FIG. 4B a conformal mold layer 42 is deposited on the inner surfaces of the concave compartments 13 of the mold 1, for example by means of spray coating. The coating, such as the conformal mold layer, may have a highly limited thickness on top of the sharp cutting edges 17, such as a thickness being in the range of 0.5-10% of an overall coating thickness. The mold layer 42 may be composed of one or more sublayers each comprising one or more materials dissolved in suitable solvents. In FIG. 4C the pharmaceutical composition 43 is loaded into the concave compartments 13 having on the top surface the mold layer 42. In one or more example methods disclosed herein, the pharmaceutical composition 43 is compacted prior to the subsequent step. In FIG. 4D the top layer 44, which may be composed of one or more sublayers each comprising one or more materials, is deposited, for example by means of spray coating, slot die coating or any other analogue method that allows for depositing thin layers of polymers on topographies that are essentially flat. The top layer 44 may be deposited on top of the pharmaceutical composition 43. In FIG. 4E discrete particles are made in the cutting step in which a planar mechanical, pneumatic, or hydraulic force/pressure is used for making the sharp cutting edges fully penetrate the mold layer 42 and top layer 44 residing on the apex of the sloped sidewalls forming the concave compartments 13, such as on the cutting edges 17, of the mold 1. The planar mechanical, pneumatic, or hydraulic force/pressure may act on the top layer 44. In FIG. 4F the cut particles 50 are released from the mold 1 directly (top part) or onto a flexible foil 45. The flexible foil 45 optionally has on the surface contacting the top layer, a material or composition that enables release of the microparticles 50 from the mold 1.

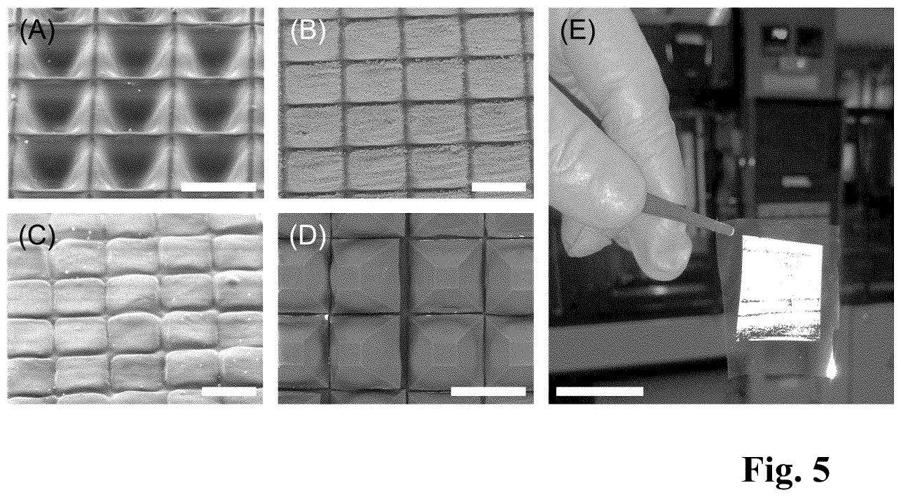

FIG. 5 shows a step-by-step illustration of the different steps involved in the disclosed method. The images (A)-(D) of FIG. 5 are scanning electron microscopy images and the scale bar of each image corresponds to 200 μm. Image (A) of FIG. 5 shows the microfabricated mold after spray coating the mold layer composed of PLGA 50:50 with a molecular weight of Mw=38-54 kDa onto the top surface of the mold 1. The PLGA may be dissolved in ethyl acetate prior to spray coating. Notice the conformal coating and the reduced thickness on the apices constituting the cutting edges. Image (B) of FIG. 5 shows a loading of the concave compartments of the mold, that have been coated with the mold layer. The compartments may, in one or more examples disclosed herein, be loaded with Barium Sulfat (BaSO₄) which can be used for X-ray detection of the microparticles. Image (C) of FIG. 5 shows a spray coated top layer on top of the loaded compartments. The coating for the top layer is in this case also PLGA 50:50 with Mw=38-54 kDa. As can be seen in image (C) FIG. 5 the microparticles appear to be swollen which is caused by a tight seal between the mold layer and the top layer in conjunction with a relatively low chamber pressure of 30-50 Pa used during inspection. Image (D) of FIG. 5 shows a plurality of discrete microparticles residing on a surface of a flexible foil with a residue-free adhesive. The particles have been cut and subsequently released from the concave compartments of the mold. The individual particles can easily be distinguished and are merely separated by a small distance. Image (E) of FIG. 5 shows a camera picture of the flexible foil containing the released particles. The release yield is almost 100 % and the particles can be scraped off and used for in vitro experiments and/or loaded inside a suitable capsule for in vivo experiments. The scale bar of image (E) of FIG. 5 corresponds to 25 mm.

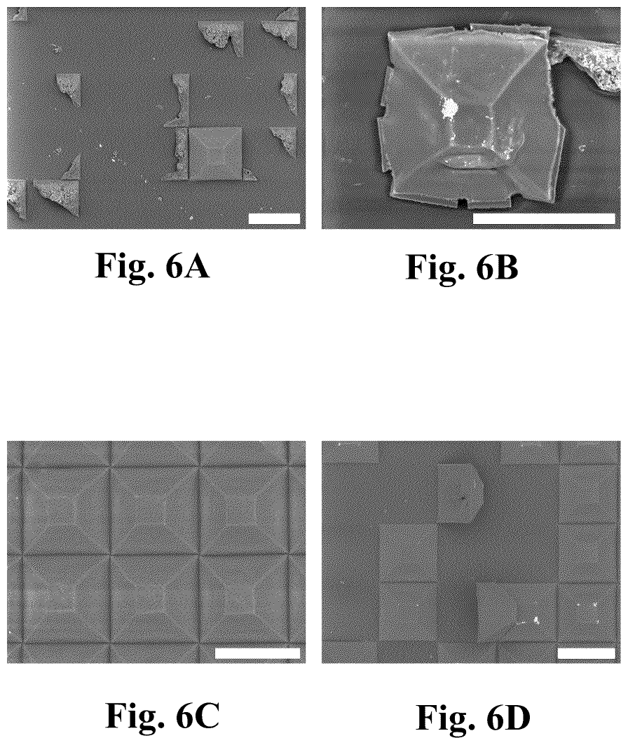

FIGS. 6A-6D show scanning electron microscopy images of produced particles illustrating the importance of having a relatively flexible top layer. The particles shown in FIGS. 6A and 6B are made using a 10-15 μm thick PLGA mold layer. The particles are loaded with indomethacin. The top layer is consisting of the enteric material Eudragit® L100. As should be evident from FIGS. 6A and 6B, the released particle yield is low and it is clearly evident that the top layer has cracked during either micro-cutting or release. In FIG. 6B, the flexible PLGA mold layer is seen covering the fractured enteric top layer. In FIGS. 6C and 6D, the top layer has been replaced by Eudragit® L100 with 20 % (w/w, PEG 6000/Eudragit® L100) PEG 6000 acting as a solid-state hydrophilic plasticizer. The mold layer is still PLGA and the particles have also been loaded with indomethacin. It is clearly evident, that having a flexible top layer dramatically increases the particle yield and as seen from the FIG. 6D, the enteric top layer is intact and there is a tight seal in the interface between mold layer and top layer. All scalebars in FIGS. 6A-6D correspond to 200 μm.

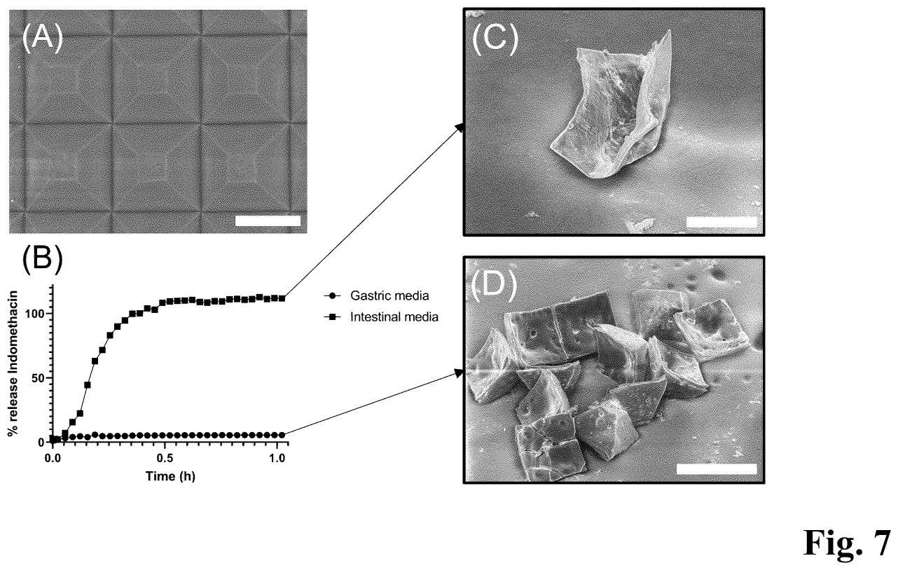

FIG. 7 shows scanning electron images and in vitro release data pertaining to an experiment with microencapsulated indomethacin as the composition. Image (A) of FIG. 7 is a scanning electron microscopy image showing released microparticles where the mold layer is 10 μm thick PLGA, the loaded compound is indomethacin, and the top layer is a 10 μm thick enteric coating consisting of Eudragit® L100 with PEG 6000 added as solid plasticizer. The sharp cutting lines separating the individual particles can be noted. The scalebar of image (A) corresponds to 150 μm. Image (B) of FIG. 7 discloses a graph showing release data from an in vitro micro-dissolution experiment. As is evident from the graph, the indomethacin is not released in a simulated gastric medium having a pH of 2. In a simulated intestinal medium at pH 6.5, the enteric coating is dissolved and 100% release is obtained within approximately 30 minutes. Image (C) of FIG. 7 shows a scanning electron microscopy image of a particle after prolonged immersion in simulated intestinal medium. Image (C) clearly illustrates that the top layer is fully dissolved, the indomethacin is released and the empty PLGA shell is still intact. The scalebar of image (C) corresponds to 100 μm. Image (D) of FIG. 7 shows a scanning electron microscopy image of an ensemble of particles after prolonged immersion in the simulated gastric medium. It is evident from image (D) that the particles are still intact. The scalebar of image (D) corresponds to 250 μm.

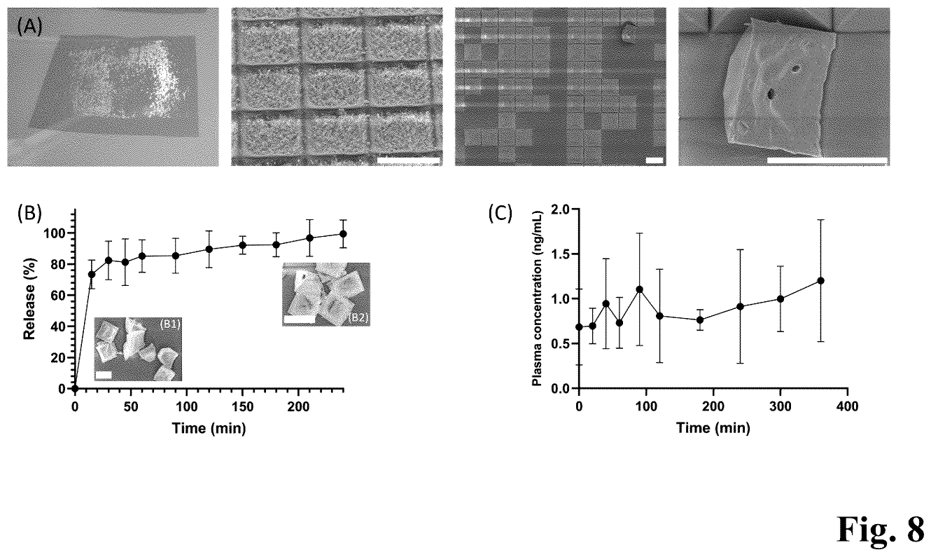

FIG. 8 shows data pertaining to the fabrication, in vitro and in vivo experiments revolving around microencapsulated delivery of the peptide drug calcitonin. Image series (A) shows a picture and scanning electron microscopy images of the produced microparticles having a PLGA mold layer, loaded with salmon calcitonin and finally coated with a flexible enteric top layer consisting of Eudragit® L100 with PEG 6000. In the leftmost picture of image series A, the compound foil is seen after releasing the produced microparticles. The scanning electron microscopy images show (from second left to rightmost image): the microfabricated mold after PLGA mold layer deposition and manual loading of the calcitonin, which is residing nicely inside the concave compartments, the produced microparticles after release onto the compound foil, and finally a zoom-in on one of the produced particles where both the PLGA mold layer and the flexible enteric top layer is visible. Notice the tight seal obtained at the interface between mold layer and top layer.

This efficiently prevents premature leakage of the loaded pharmaceutical composition. All scalebars of image series A correspond to 200 μm. Image (B) discloses a graph showing a triggered release profile of microencapsulated calcitonin in a phosphate buffer of pH 6.8. The release was quantified using high-performance liquid chromatography and the measurements were continued for 4 h. Within the first 30 minutes most of the calcitonin (approximately 80%) is released in a rapid manner. Hereafter, a slow release is evident until the measurements were terminated at t=240 minutes. The insets in image (B) show scanning electron microscopy images of the particles after 1 hour immersion in gastric medium of pH 1.6 (inset B1) and after the in vitro release experiment conducted at pH of 6.8 (inset B2). It is clearly evident, that the microparticles are intact after the immersion in the gastric-like fluid of low pH whereas the flexible enteric top layer has been totally dissolved after the release experiment in the phosphate buffer used for mimicking the small intestinal environment. The scalebars in (B1) and (B2) correspond to 200 μm.

Image (C) discloses a graph showing an average (n=6) blood plasma concentration of calcitonin in rats after dosing with the produced microparticles. The plasma concentration of the peptide drug is relatively low but quite stable over a time period of 6 hours at which point the blood sampling was terminated. This preliminary data shows that the disclosed fabrication method can be used for making microencapsulated pharmaceutical compositions suitable for e.g., oral delivery of peptide drugs.

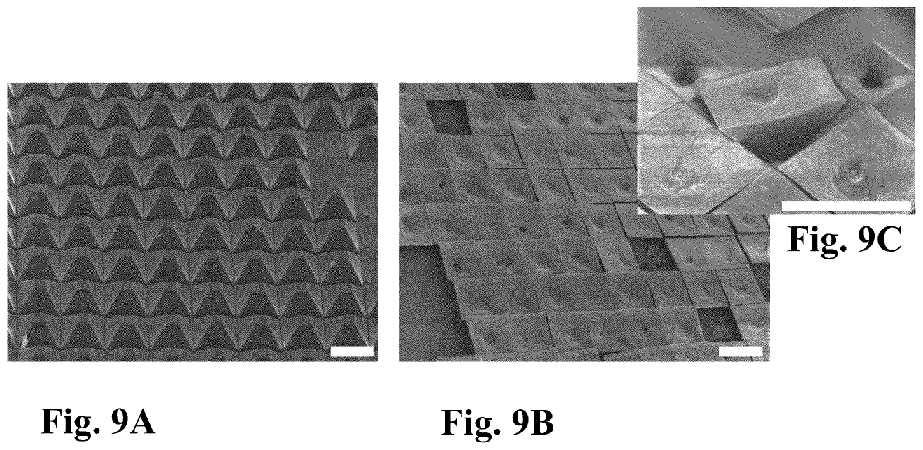

FIGS. 9A-9C show scanning electron microscopy images of produced microparticles after micro-cutting and release onto a compound foil, such as a mildly adhesive compound foil. FIG. 9A shows a vast amount of microparticles residing on the compound foil. The particles consist of a PCL|PLGA dual mold layer, the particles are loaded with spray dried paracetamol (BCS class I drug) and have finally been coated with an enteric top layer consisting of Eudragit® FL 30 D-55 diluted in acetone. FIGS. 9B and 9C show the produced particles after lifting them off onto an adhesive pad for visual inspection of the top layer. It is evident from FIGS. 9B and 9C, that the top layer is covering the loaded pharmaceutical composition nicely with only a limited number of small pinholes visible. Furthermore, the tight seal in the interface between the mold layer and top layer is clearly seen in FIG. 9C. All the scalebars of FIGS. 9A-9C correspond to 200 μm.

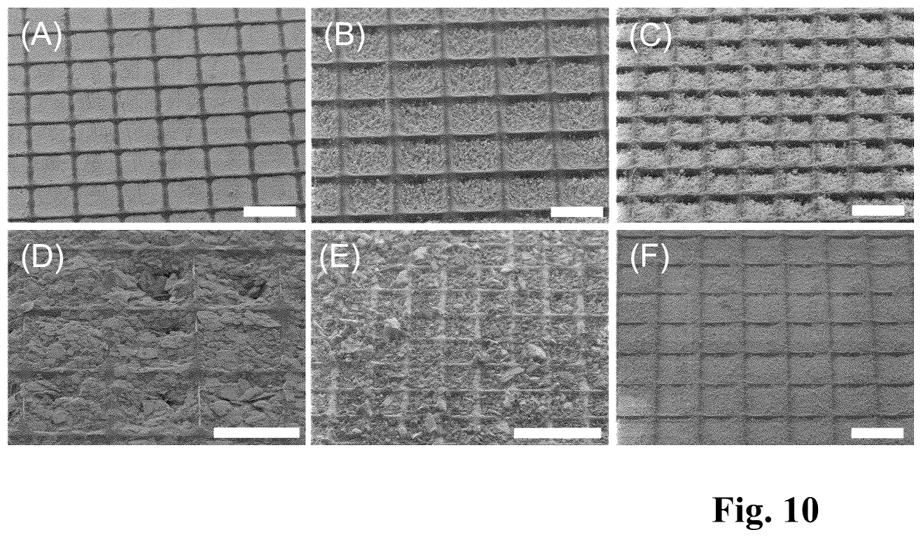

FIG. 10 shows scanning electron microscopy images (A)-(F) of the microfabricated mold after spray coating a PLGA mold layer onto the mold and loading different pharmaceutical compositions. Image (A) shows a loading of the compartments of the mold with solid state BaSO4 used for X-ray tracking of the microparticles both ex vivo and in vivo. As is evident from image (A), the BaSO4 material packs nicely and fills up the concave compartments. The scale bar of image (A) corresponds to 250 μm. Image (B) shows a loading of the compartments of the mold with solid state calcitonin. Solid state calcitonin is a small polypeptide with a molecular weight of 3.4 kDa used in the treatment of osteoporosis. Calcitonin is a BCS class III drug with associated low permeability but high solubility. The scale bar of image (B) corresponds to 250 μm. Image (C) shows a loading of the compartments of the mold with solid state indomethacin. Solid state indomethacin is a nonsteroidal anti-inflammatory drug (NSAID) used for treatment of acute pain and relieve symptoms of arthritis or gout. Indomethacin is a BCS class II drug characterized by high permeability and low solubility. The scale bar of image (C) corresponds to 300 μm. Image (D) shows a loading of the compartments of the mold with a pharmaceutical paste composed of paracetamol, talcum, glycerol, and ultrapure water. Notice the large flat flakes. The paste adapts nicely to the concave compartments. The scalebar of image (D) corresponds to 250 μm. Image (E) shows a loading of the compartments of the mold with solid state paracetamol after grinding to reduce the particle size of the paracetamol. It is clearly evident from image (E) that large particles are still present. Some particles are almost as large as the concave compartments of the mold. The scalebar of image (E) corresponds to 500 μm. Image (F) shows a loading of the compartments of the mold with spray dried paracetamol. Upon comparing image (F) to image (E) a dramatic reduction in particle size is evident. The spray dried formulation packs nicely inside the concave compartments. The scalebar of image (F) corresponds to 300 μm.

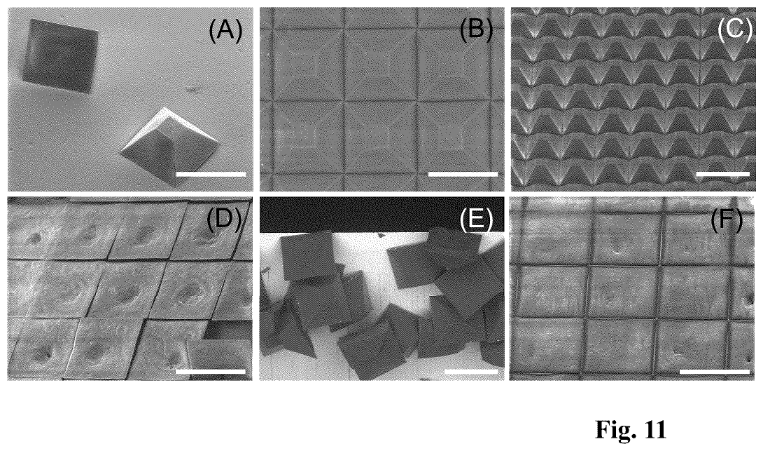

FIG. 11 shows scanning electron microscopy images of particles featuring different mold layers, loaded compositions and top layers but all illustrating the monodisperse nature of the particles produced using the method presented in the current disclosure. Image (A) shows particles where both the mold layer and top layer is PLGA and the loaded pharmaceutical composition is spray dried paracetamol. Image (B) shows particles featuring a PLGA mold layer, loaded with indomethacin and top coated using Eudragit L100 with solid PEG plasticizer. Image (C) shows particles having a mold layer composed of PCL|PLGA, loaded with spray dried paracetamol and having an enteric top coating of Eudragit L100 with solid PEG plasticizer. Image (D) shows particles with a PCL|PLGA mold layer, loaded with spray dried paracetamol and having a flexible enteric top coating composed of Eudragit FL 30D-55 . Image (E) shows particles on a razor blade after release. The particles are loaded with BaSO₄ and both the mold and top layer is PLGA. Image (F) shows fully enteric particles where the mold and top layer are composed of Eudragit L100 with solid PEG plasticizer. The particles are shown immediately after cutting. The scale bars in all pictures correspond to 200 μm.

### EXAMPLES

### Example 1: Microfabricated Silicon Mold

A silicon mold (as shown in FIG. 2) featuring close-packed concave compartments having an inverse regular pyramidal frustum geometry and a sidewall taper angle of approximately 50-55° was produced by conventional top-down processing of single-side-polished silicon (thickness=525±20 μm, Siergert Wafer GmbH, Aachen, Germany). Initially a 1 μm thick SiO₂ layer was grown by wet thermal oxidation at 1100° C. in a dedicated horizontal furnace (Tempress, Vaassen, Netherlands). This layer was used as a hard mask in the subsequent processing steps. By flat aligned UV lithography (MLA150 WM I, Maskless Aligner, Heidelberg Instruments) in 1.5 μm thick positive resist AZ®5214 E (MicroChemicals GmbH, Ulm, Germany) an etching mask was made in the photoresist. This pattern was transferred to the underlying oxide layer by means of dry etching using an Advanced Oxide Etching (AOE, STS MESC Multiplex ICP) tool. The oxide etch was carried out with 5 sccm C₄F₈, 4 sccm H₂ and 174 sccm He as the reactive gasses. The etch was conducted using a chamber pressure of 4.0 mTorr, a coil power of 1300 W and a platen power of 200 W. After the dry etch, the remaining photoresist was stripped using oxygen plasma ashing (300 Semi Auto Plasma Processor, PVA TePla America Inc.) followed by immersion in concentrated H₂SO₄ with (NH₄)₂S₂O₈ salt added. This solution is commonly referred to as 7-up. The temperature of the wet chemical bath was kept at 80° C. during the immersion. To achieve sidewall taper angles of approximately 50-55°, the hard masked silicon substrate was subject to a wet anisotropic etch in 28 wt % KOH heated to 80° C. The etch was terminated when the compartment depth was 100 μm. After the KOH etch, the remaining SiO₂ was stripped in buffered hydrofluoric acid (12 vol % HF with NH₄F) and the substrate was subject to a 10 min clean at 80° C. in 7-up to remove trace contaminants. The sharp cutting edges were obtained in a dry isotropic etch using an Advanced Silicon Etching (ASE, STS MESC Multiplex ICP) tool. The reactive gasses were SF₆ (230 sccm) and O₂ (23 sccm) and the substrate temperature was kept at 20° C. The manual pressure setting was 87.7% and the coil power and platen power was 2800 W and 19 W, respectively. After the ASE etch, potential trace contamination was removed by oxygen plasma ashing followed by 7-up immersion. Before using the microfabricated mold for particle production, the top surface of the silicon mold was coated with a thin anti-stick layer (MVD 100 Molecular Vapor Deposition System, Applied Microstructures Inc.) constituted by a conformal monolayer of 1H,1H,2H,2H-perflourodecyltrichlorosilane (FDTS). The anti-stick layer reduces the surface energy of the concave compartments which effectively decreases the adhesion between the produced microparticles and the mold. This in turn alleviates issues pertaining to particle stiction during the release cycle. The microfabricated mold was characterized using a combination of optical microscopy (Nikon ECLIPSE L200, Nikon Metrology Europe NV, Leuven, Belgium), vertical scanning interferometry (PLu Neox 3D Optical Profiler, Sensofar Metrology, Terrassa, Spain) and scanning electron microscopy (SEM, Zeiss Supra 40 VP). The example produced mold features concave compartments with an average depth of 103 μm, a distance between cutting edges of 200 μm and a sidewall taper angle of approximately 55°.

### Example 2: Full Shell PLGA Particles Loaded With BaSO₄ for X-ray Tracing

Microparticles, having a regular pyramidal frustum shape, where made using a mold with a compartment depth of 100 μm and a distance between cutting edges of 200 μm (refer to FIG. 5). Initially a mold layer composed of PLGA 50:50 (Resomer® RG 504, 38-54 kDa, Merck (previously Sigma-Aldric), Darmstadt, Germany) was deposited onto the anti-stick coated mold. The PLGA was dissolved in ethyl acetate by stirring and a 1 wt % solution was loaded in the ultrasonic spray coating machine (ExactaCoat, Sono-Tek, Milton, NY, USA). A micro-bore fitted 120 kHz ultrasonic Vortex nozzle was used for spray coating. The Vortex nozzle features a circulating cladding air which improves conformality during deposition of the mold layer. The spray coating of the mold was performed using linear passages across the mold. A polymer solution infusion rate of 1.5 mL/min was used and the ultrasonic generator power was fixed at 1.5 W. The cladding air pressure was set to 0.03 bar and the nozzle speed was maintained at 50 mm/s. The nozzle was programmed to pass over the sample with a nozzle-to-mold distance of 50 mm using a cross-like passage pattern (such as two linear passages followed by two linear passages where the translation orientation was orthogonal to the first set of passages). A total of 75 loops (consisting of 4 linear passages) was used to deposit an approximately 12.5 μm thick mold layer. During spray coating, the hot plate temperature was kept at 35° C. The resulting PLGA mold layer was highly conformal and almost entirely pinhole free. The thickness was measured on a flat anti-stick coated silicon reference sample using stylus profilometry (Dektak 150, Veeco, Plainview, NY, USA). Following the mold layer coating, the concave compartments were loaded with precipitated and grounded BaSO₄ (99% pure BaSO₄, Thermo Fisher Scientific, Waltham, MA, USA) which allows for particle tracking via planar X-ray imaging or computed tomography scanning. The powder was loaded manually using a fine brush which resulted in an excellent loading of the solid state material. After loading the radiopaque material, the top layer was deposited by ultrasonic spray coating. The top layer, which effectively encapsulates the loaded BaSO₄, was coated using the exact same parameters as used for coating the mold layer. Following the microencapsulation, discrete particles were obtained by cutting which was done using a conventional hot embosser (Collin Press P300 SV, Collin Lab & Pilot Solutions GmbH, Maitenbeth, Germany). The hot embosser delivers a parallel-plate hydraulic pressure which ensures that the microscopic pressure associated with the sharp cutting edges exceeds the ultimate tensile strength of the PLGA layer used for the mold and top coating. During the cutting procedure, the mold was pressed against a tough and deformable aluminum foil using a hydraulic pressure of 10 bar for 60 s. The actual pressure experienced by the mold was 1963N/cm² . After the cutting step, the discrete particles were released onto a compound foil having an overall thickness of 70-75 μm. The foil (Nitto SWT 20+R, Nitto Denko Corporation, Osaka, Japan) consists of flexible polyvinyl chloride with one side coated with a silicone release coating and the opposite side (onto which the particles are released) coated with a pressure sensitive acrylic-based adhesive that leaves no residues on the particles when they are detached from the carrier foil. The release was conducted in the hot embosser using a hydraulic pressure of 10 bar for 60 s.

### Example 3: Particles With Brittle Top Layer and Why It Should Be Avoided

To illustrate the potential negative impact of utilizing brittle materials in the top layer during production of microencapsulated pharmaceutical compositions using the disclosed method, the current example considers production of indomethacin loaded microparticles consisting of a 10-15 μm thick PLGA mold layer (refer to example 2 for details) and two different top layers. Initially the mold layer was spray coated on the microfabricated mold and afterwards, finely ground indomethacin was loaded manually into the concave compartments. Two samples were prepared: one sample where the top layer was consisting solely of the enteric material Eudragit® L100 and one sample where polyethylene glycol (PEG 6000, Merck KGaA, Darmstadt, Germany) was added as a solid hydrophilic plasticizer to modify the mechanical properties of the top layer. In both cases, spray coating was employed for deposition and the details have been highlighted in example 2 and 4. In FIG. 6, scanning electron microscopy images acquired after micro-cutting and release are presented. It is clear from the images in the top row, that the particle yield is almost zero when employing Eudragit® L100 as top layer. The enteric layer simply cracks and detaches from the mold layer. Remaining and fragmented portions of the mold layer with substantial traces of indomethacin are seen residing on the top surface of the compound foil used for particle release after the micro-cutting. Preliminary attempts at incorporating liquid plasticizers (the main focus was on dibutyl sebacate (DBS) and triethyl citrate (TEC)) commonly used in the pharma industry was not resulting in detectable changes in the elastic properties of the top layer. This may be a result of the intrinsically hydrophobic properties of DBS and TEC which prevented sufficient incorporation into the hydrophilic Eudragit® L100. Switching to the hydrophilic solid plasticizer PEG 6000 had an immediate effect. With 20 wt % PEG 6000 added to the Eudragit® L100, the brittleness of the top layer was reduced and the produced particle yield increased dramatically. As should be evident from the scanning electron microscopy images in the bottom row of FIG. 6, intact microparticles were now easily produced and released onto the compound foil with a high yield. There was no sign of cracks in the enteric top layer which forms an effective seal in the interface between the mold layer and top layer thereby ensuring microencapsulation of the loaded indomethacin.

### Example 4: In Vitro Release Experiments With Indomethacin

In order to test the produced microparticles with a suitable pharmaceutical composition, particles consisting of a PLGA mold layer was loaded with the BCS class Il drug indomethacin (>98% pure, Tokyo Chemical Industry, Japan) and finally coated with a flexible enteric top layer (refer to FIG. 7). Particles having a regular pyramidal frustum shape were produced using an anti-stick coated silicon mold with a compartment depth of 100 μm and a distance between cutting edges of 200 μm. As outlined in example 2, the microfabricated mold was subject to a mold layer coating using ultrasonic spray coating with a microbore-fitted 120 kHz Vortex nozzle (Sono-Tek, Milton, NY, USA). The mold layer, having an approximate thickness of 10-15 μm, was composed of PLGA 50:50 (Resomer® RG 504, 38-54 kDa, Merck (previously Sigma-Aldrich). After the PLGA coating step, indomethacin was loaded inside the concave compartments using a fine brush. Prior to loading, a fine-grained amount of indomethacin was prepared using a mortar. After the drug loading, an enteric coating composed of 1% w/v Eudragit® L100 (Evonik Industries GmbH, Essen, Germany) dissolved in isopropanol with 20% w/w (plasticizer/Eudragit® L100) polyethylene glycol (PEG 6000, Merck KGAA, Darmstadt, Germany) added as a solid plasticizer. The addition of a solid plasticizer effectively reduces the intrinsic brittleness of the enteric top layer, thereby allowing for a high yield particle production. The top layer was spray coated using a generator power of 1.5 W, a solution infusion rate of 1.5 mL/min, a cladding air pressure of 0.025 bar, a nozzle translation speed of 50 mm/s, a nozzle-to-mold distance of 50 mm and the hot plate was kept at 40° C. An enteric top layer having a thickness of approximately 10 μm was deposited using 50 loops, each consisting of 4 linear passages across the mold. Discrete particles were obtained using a conventional hot embosser (Collin Press P300 SV, Collin Lab & Pilot Solutions GmbH, Maitenbeth, Germany) for cutting through the mold layer and top layer. Finally, the particles were released onto a compound foil having an overall thickness of 75 μm (Nitto SWT 20+R, Nitto Denko Corporation, Osaka, Japan). In order to test the encapsulation efficiency and drug release, an in vitro experiment was conducted. Using a microDISS Profiler™ (Pion incorporated, Billerica, MA, USA), the drug release was studied in gastric medium of pH 2 and subsequently in Fasted State Simulated Intestinal Fluid (FaSSIF, Biorelevant. com, London, United Kingdom) at pH 6.5. The microDISS Profiler™ was equipped with in situ fiber-optic UV probes with a path length of 5 mm and the absorbance was measured in the range of 310-320 nm. The temperature was set at 37° C. and a rotation speed of 200 rpm. Prior to the release experiment, a standard curve was constructed by adding aliquots of a stock solution consisting of indomethacin in methanol to the release medium. Particles experiencing prolonged immersion (i.e., more than 24 h) in gastric medium did not release any significant amount of indomethacin and the particles were intact as confirmed by scanning electron microscopy inspection. Particles immersed in the FaSSIF had a characteristic fast and full release within a time span of 30 minutes. Upon post release inspection using scanning electron microscopy, the particles were empty and the only thing remaining was the PLGA mold layer.

### Example 5: In Vitro and In Vivo Data Pertaining to Calcitonin Delivery in Rats

In order to test the disclosed fabrication method in an in vivo setting, microparticles consisting of a 10-15 μm thick PLGA mold layer, loaded with finely ground salmon calcitonin powder and finally coated with an enteric top layer consisting of Eudragit® L100 with 20 wt% PEG 6000 were produced. Initially the PLGA mold layer was deposited on the microfabricated mold as described in example 2. Hereafter, finely ground calcitonin powder was loaded manually into the concave compartments using a fine brush and the flexible enteric top layer was deposited to fully encapsulate the calcitonin (details found in example 4). Calcitonin is a small peptide drug having a molecular weight of 3.4 kDa. Calcitonin is used in the treatment of osteoporosis, and it is classified as a BCS class III drug with low permeability and high solubility. Currently, there are no commercial oral dosage forms available on the market-mainly due to problems in obtaining a sufficient systemic uptake when dosing the peptide drug orally. The discrete microparticles where produced by micro-cutting and finally released onto the mildly adhesive compound foil. As seen in FIG. 8 (A), the calcitonin was nicely encapsulated in the microparticles. Prior to the in vivo experiments a release study was conducted in vitro to investigate the properties of the microparticles. Initially, the particles were immersed in a gastric medium (0.02 M HCl of pH 1.6) for 1 hour to investigate whether the particles remained intact at gastric pH. As seen from inset (B1) in FIG. 8 the particles were still intact after the gastric step. Next the microparticles were poured into glass vials and 10 mL of phosphate buffer at pH 6.8 was added and stirred at 100 rpm. The calcitonin release was quantified by high-performance liquid chromatography (HPLC) for a duration of 4 hours. HPLC is a chromatographic method where solubilized compounds allow for molecular separation. By the technique, it is possible to have quantitative analysis of which components and in which concentrations they are contained in a sample. As seen from the release graph in FIG. 8 (B), most of the calcitonin (approximately 80%) is released in a fast manner within the first 30 minutes. Hereafter, the remaining calcitonin is released at a slow rate until the experiment was terminated at t=240 minutes. To verify that the enteric top layer was dissolved, and the calcitonin released, scanning electron microscopy images (included in FIG. 8 (B2)) were acquired after the in vitro experiment. Here it is clearly evident that the enteric coating has been fully dissolved and only traces of calcitonin are left on the PLGA mold layer. For the in vivo study, the microparticles were loaded into size 9 gelatin capsules that were weighted before and after loading in order to calculate the amount of calcitonin administrated to the rats. A total of 6 Sprague Dawley rats weighting between 326-353 g were orally administered with one capsule containing calcitonin-loaded microparticles. Blood samples were taken in the teil vein over 6 hours. The plasma samples were analyzed using an ELISA kit. The results show that small amounts of the calcitonin was absorbed into the blood of the rats from the microparticles. With a control of non-encapsulated calcitonin powder in a gelatin capsule nothing was absorbed due to degradation and very low permeation of calcitonin over the intestinal membrane. Therefore, we can conclude from these preliminary studies, that dosing calcitonin in the produced microparticles results in oral absorption over the intestinal membrane which is a very promising result in terms of using the microparticles for oral peptide/protein delivery in the future.

### Example 6: Particles With Two Materials (PCL and PLGA) in the Mold Layer

In order to illustrate that the disclosed method can indeed be used for making microparticles where multiple materials are used during deposition of the mold and/or top layers, particles featuring a mold layer composed of PCL (Mw=80 kDa) and PLGA (Mw=38-54 kDa) were produced. The two layers were deposited in a sequential manner by ultrasonic spray coating on the microfabricated mold. The PCL layer was added as a mold interface layer to alleviate particle release after micro-cutting. As PCL exhibits a low surface free energy, the adhesion to the microfabricated mold is decreased as compared to e.g., PLGA. The PCL layer was deposited by spray coating a 0.5 wt% solution of PCL dissolved in dichloromethane (DCM). The following parameters were used during spray coating: hotplate temperature=25° C., ultrasonic generator power=1.0 W, liquid infusion rate=1.5 mL/min, cladding air pressure=0.03 bar, nozzle translation speed=50 mm/s and a nozzle-to-mold distance of 50 mm. The PCL layer, having a final thickness of approximately 7.5 μm, was then subject to another coating step in which 1.0 wt % PLGA in ethyl acetate was spray coated using a slightly increased hot plate temperature of 45° C. The remaining parameters for coating PLGA have been included in example 2. After the dual mold layer deposition, the concave compartments were loaded with spray dried paracetamol (low grain size, BCS class I drug). Finally, a top layer consisting of 1 vol % Eudragit® FL 30 D- 55 in acetone was deposited. The following parameters were used during spray coating: hotplate temperature=30° C., ultrasonic generator power=1.0 W, liquid infusion rate=1.5 mL/min, cladding air pressure=0.025 bar, nozzle translation speed=50 mm/s and a nozzle-to-mold distance of 50 mm. The specific type of Eudragit® has a built-in plasticizer which effectively ensures that the top layer is flexible enough to comply with the micro-cutting procedure. After the top layer deposition, the microparticles were cut and released onto a mildly adhesive compound foil. As seen in FIG. 9, both the mold layer and top layers are intact and pinhole free, thereby efficiently encapsulating the loaded drug substance. It is also evident, that the flexible enteric top layer is tightly sealed to the mold layer along the periphery of the micro-cut particles.

### Example 7: Suitable Formulations for the Pharmaceutical Composition

Clearly, when considering the production of microencapsulated solid or semi-solid state pharmaceutical compositions, it is important that the pharmaceutical composition itself complies with all steps in the microencapsulation process. Within the context of the present disclosure, this implies that especially the maximum spatial feature size associated with the different constituents of the pharmaceutical composition is kept small in comparison to the actual microparticle size. When considering the production of large or intermediate sized particles with hydrodynamic diameters of several hundreds of micrometers, this would usually not represent a problem. However, when scaling down the microparticle size into a regime where the particles could be feasible for nasal or pulmonary delivery things change. For nasal delivery the mean particle size should be between 30-120 μm and for pulmonary delivery sizes in the range from 1-20 μm are normally feasible. In the present disclosure preprocessing of different drugs have been carried out to facilitate loading into the shell-layer-coated compartments of the microfabricated mold. In most cases simple grinding using a mortar has been sufficient for reducing the intrinsic particle size of the drugs prior to loading. In FIG. 10, scanning electron microscopy images showing loaded substances inside the concave compartments of the mold are presented. Prior to loading, a 10-15 μm thick mold layer, such as a PLGA layer, was deposited by ultrasonic spray coating. Regular powders such as the BaSO₄ packs perfectly inside the concave compartments as seen in FIG. 10 (A). When considering e.g., amorphous substances such as peptide drugs and certain other conventional drugs, a similar packing efficiency would be expected unless the drug is highly hygroscopic. This could lead to problems during loading due to an increased tackiness of the drug when exposed to ambient humidity. As seen in FIG. 10 (B) and (C), showing loaded calcitonin and indomethacin, a simple grinding allows for reducing the particle size prior to loading thereby enabling efficient loading of the concave compartments. A similar approach can be attempted when preprocessing crystalline or semi-crystalline compositions such as e.g., paracetamol. As seen in FIG. 10 (E), grinding has resulted in a fine powder but large particles/flakes having sizes comparable to or exceeding the size of the concave compartments are clearly evident. In this case, more advanced preprocessing of the pharmaceutical composition may be needed in order to facilitate the microencapsulation. A range of preprocessing methods have been included in the section “Pharmaceutical composition”. In the case of paracetamol, a spray dryer machine (Mini Spray Dryer B-290, BUCHI, Sankt Gallen, Switzerland) was used to produce a powder in which the maximum grain/particle size was much smaller than the concave compartments of the microfabricated mold. For that purpose, the following parameters were used when spray drying: aspirator=28 m³/h, volume flow=357 L/h, pump rate=5 mL/min, inlet and outlet temperature=100° C. and 45° C., respectively. As seen in FIG. 10 (F), the spray dried paracetamol powder is easily packed with high efficiency inside the compartments. The current disclosure mainly focuses on microencapsulation of solid-state pharmaceutical compositions, but also semi-solid compositions can be encapsulated using the fabrication method. Semi-solid compositions include slurries and/or pastes and again the ultimate demand is that the maximum particle size of any constituent in the compositions must be low in comparison to the size of the concave compartments of the microfabricated mold. In FIG. 10 (D) we have included an image showing loading of a pharmaceutical paste composed of paracetamol and talcum in a proportion of 1:2, respectively dispersed in a mixture of ultrapure water and glycerol in a proportion of 1:1.

### Example 8: Monodispersity of the Produced Particles

The fabrication method presented in the current disclosure employs a microfabricated mold comprising concave compartments where the design parameters including compartment depth, sidewall taper angle and distance between opposing cutting edges are all subject to minimal variations (i.e. 1-2% or below). This infers that both the geometry and inner volume associated with the concave compartments are subject to minimal variations. For the mold presented in example 2, the compartment depth is 103 μm±0.4 μm (average±std, N=3 measurements), the taper angle is 55° and the distance between cutting edges is 200 μm±0.75 μm (average±std, N=5 measurements). The particles of the current disclosure are produced by coating a mold layer onto the mold, loading and optionally compacting a pharmaceutical composition inside the compartments, coating a top layer and finally cutting out the particles. During the cutting process, the particles are compacted as the cutting edges penetrate slightly into the backing layer. This infers that the height of the produced particles may be subject to a small decrease as compared to the depth of the concave compartments on the mold. In addition, the top layer may assume a slightly concave morphology. The monodispersity of the particles produced using the method in the current disclosure, pertains to the size or conversely the volume and geometry of the produced particles. As the particles are produced using a mold where the geometry and inner volume of the concave compartments is subject to minimal variations, particles with a narrow size distribution is expected. In FIG. 11, particles featuring different mold layers, loaded compositions and top layers have been included. In FIG. 11 (A), (D) and (E), particles are seen after collection from the carrier foil. Here, both the mold layer and top layer are evident, and the slightly concave morphology of the top layer is primarily distinguished in (D). FIG. 11 (B) and (C) show particles residing on the carrier foil. Here the mold layer and the thin cutting lines separating individual particles are seen. The geometry and volume of the particles are subject to minimal variations thus substantiating the monodisperse nature of particles produced using the method presented in the current disclosure. In FIG. 11 (F) particles are seen residing in the mold after cutting and prior to release. The cutting edges of the mold are easily distinguished and the top layer appears to be relatively flat and homogeneous. It is important to underline, that the monodispersity of the produced particles relies not only on the mold but also on the pharmaceutical composition. As outlined in the section “Pharmaceutical composition” the loaded composition must fulfill certain requirements when it comes to the microscale morphology (i.e. the spatial size of the different constituents). Furthermore, the composition must also be loaded evenly into the concave compartments to avoid substantial deviations in the volume of the produced particles. As seen in FIG. 11, where the compositions are composed of constituents with a low grain size, even manual loading of the compartments allows for producing particles that by inspection exhibit the same size and geometry. The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another.

Note that the words “first”, “second”, “third” and “fourth”, “primary”, “secondary”, “tertiary” etc. are used here and elsewhere for labelling purposes only and are not intended to denote any specific spatial or temporal ordering. Furthermore, the labelling of a first element does not imply the presence of a second element and vice versa.

It is to be noted that the word “comprising” does not necessarily exclude the presence of other elements or steps than those listed.

It is to be noted that the words “a” or “an” preceding an element do not exclude the presence of a plurality of such elements.

Although features have been shown and described, it will be understood that they are not intended to limit the claimed disclosure, and it will be made obvious to those skilled in the art that various changes and modifications may be made without departing from the scope of the claimed disclosure. The specification and drawings are, accordingly to be regarded in an illustrative rather than restrictive sense. The claimed disclosure is intended to cover all alternatives, modifications, and equivalents.

1. A method for preparing microparticles for drug delivery, the method comprising:
i) providing a mold comprising multiple individual open compartments arranged on a first surface of the mold, wherein the open compartments are separated from each other by one or more edges, wherein each edge has a width of 5 μm or less,
ii) coating the first surface of the mold with a mold layer comprising a biocompatible and/or biodegradable material having a first release characteristic at a first condition,
iii) loading the compartments coated with the mold layer with a pharmaceutical composition comprising a drug substance,
iv) coating the first surface of the mold with a top layer comprising a biocompatible and/or biodegradable material having a second release characteristic at the first condition for provision of closed microparticles in the multiple individual compartments, and
v) separating the closed microparticles into discrete microparticles by cutting the mold layer and the top layer using the one or more edges.

2-18. (canceled)

19. The method according to claim 1, wherein the second release characteristic is different than the first release characteristic.

20. The method according to claim 1, wherein the second release characteristic is the same as the first release characteristic.

21. The method according to claim 1, wherein each compartment has a pyramidal shape.

22. The method according to claim 1, wherein each compartment has a base area and a top area and wherein the base area of each compartment is larger than or equal to the top area.

23. The method according to claim 22, wherein a surface area of the base area is equal to or less than 500×500μm².

24. The method according to claim 22, wherein an orthogonal distance from the base area to the top area of each compartment is 500 μm or less.

25. The method according to claim 21, wherein a cross section of each compartment has a trapezoid shape.

26. The method according to claim 25, wherein the trapezoid has a top side, a bottom side and connecting sides that connect the top side with the bottom side, wherein the angle between the top side and the connecting side is less than 90 degrees.

27. The method according to claim 1, wherein the first condition is at a pH in the range of 5-7 and a temperature in the range of 36-38 degrees C.

28. The method according to claim 1, wherein the biocompatible and/or biodegradable material in the mold layer and/or top layer is a film-forming polymer selected from the group consisting of polyurethane (PUR), polycaprolactone (PCL), polylactic acid (PLA) in all stereoisomeric forms, polyglycolic acid (PGA), co-polymers of PLA and PGA known as poly lactic-co-glycolic acid (PLGA), polyvinylpyrrolidone (PVP), polyvinyl alcohol (PVA), polyethylene glycol (PEG), polyacrylic acid (PAA), hydroxypropyl methylcellulose (HPMC) and ethylcellulose.

29. The method according to claim 1, wherein the material of the mold and/or top layer comprises a film-forming polymer selected from the group consisting of co-polymers based on polymethacrylic acid and methacrylates, ethyl acrylate and methyl acrylate, co-polymers of acrylic and methacrylic acid esters, hydroxypropyl methylcellulose phthalate, cellulose acetate phthalate, polyvinyl acetate phthalate and mixtures thereof.

30. The method according to claim 1, wherein the material of the mold layer and/or top layer comprises a film-forming polymer selected from the group consisting of cellulose, cellulose derivatives, methylcellulose, hydroxypropylcellulose, hydroxypropyl methylcellulose, and mixtures thereof.

31. The method according to claim 1, wherein the material of the mold layer and/or top layer comprises a plasticizer.

32. The method according to claim 1, wherein the mold comprises silicon.

33. The method according to claim 1, wherein the first release characteristic and/or second release characteristic is one or more of a water solubility, a dissolution rate, a degradation rate, or a permeability of the loaded pharmaceutical composition.

34. The method according to claim 1, wherein the discrete microparticles are monodisperse.

35. The method according to claim 1, wherein the discrete microparticles have a pyramidal shape.
